(19) United States
(12) Patent Application Publication
(10) Pub. No.: US 2024/0198177 A1
(43) Pub. Date: Jun. 20, 2024

(54) EXERCISE INSTRUCTION AND FEEDBACK SYSTEMS AND METHODS

(71) Applicant: Peloton Interactive, Inc., New York, NY (US)

(72) Inventors: Christopher Richard KRUGER, New York, NY (US); Michael Skyler ERICKSON, New York, NY (US)

(21) Appl. No.: 17/909,311

(22) PCT Filed: Mar. 3, 2021
(86) PCT No.: PCT/US2021/020743

§ 371 Date: Sep. 2, 2022

Related U.S. Application Data
(60) Provisional application No. 62/985,149, filed on Mar. 4, 2020.

(51) Int. Cl. A63B 24/00

(52) U.S. Cl. CPC A63B 24/0075; A63B 24/0006; A63B 2024/0068; A63B 2220/806

(57) ABSTRACT

Systems and methods include an exercise device receiving a sequence of camera images of a user performing an exercise, identifying a plurality of anatomical points of the user based at least in part on the sequence of camera images, determining a user performance level based, at least in part, on an analysis of the plurality of anatomical points; and providing corrective feedback to the user when the user performance level fails to satisfy performance criteria for the exercise. A workout device includes a display device for displaying exercise content and a housing with a recessed portion and a camera arm configured to be inserted in the recessed portion of the housing in a first position and partially inserted in the recessed portion of the housing in a second position.

### CROSS-REFERENCE TO RELATED APPLICATIONS

The present application is a national phase entry of international patent application No. PCT/US2021/020743, filed Mar. 3, 2021, which claims the benefit of and priority to U.S. Provisional Application No. 62/985,149, filed Mar. 4, 2020, which are hereby incorporated by reference in their entirety.

### TECHNICAL FIELD

The disclosed subject matter relates generally to systems and methods for exercise instruction and feedback, and in some embodiments, to home-based exercise systems and methods such as mat-based exercise systems.

### BACKGROUND

The home exercise market is growing with people looking for convenient and effective ways to exercise in their own homes. One drawback is that in-home exercise equipment tends to be bulky and cumbersome, occupying a larger than desired amount of space in a person's home. Another drawback is that the user can feel detached and unmotivated when working out alone. As a result, new in-home exercise equipment and exercise media have been developed to provide the user with a personalized and engaging in-home experience through equipment that is convenient for use in the home.

In some exercise systems, exercise applications are provided that allow the user to stream and view exercise content, such as an exercise class led by an instructor. Although exercise videos can make working out more enjoyable, the user does not receive expert feedback while exercising like the user would exercising in-person at a gym. When exercising in a gym with a personal trainer or in an exercise class led by an instructor, a fitness professional can provide real time feedback on the exerciser's performance and provide motivational cues to keep the exerciser engaged and on track. For example, a person may perform an exercise with incorrect form, which can diminish the effectiveness of the training and potentially risk an injury. Additionally, a user may be unmotivated to finish a workout or to maintain a desired intensity level when exercising alone. By contrast, a live instructor or coach can provide motivation and encouragement during the workout, leading to a better overall exercise experience.

In view of the foregoing, it is desirable to provide new exercise systems, methods, and media for presenting exercise content to user with instructor feedback.

### SUMMARY

The present disclosure is directed to improved systems and methods for presenting exercise content (e.g., video, audio, data) to a user, including feedback on the user's performance. In some embodiments, a device for presenting exercise videos with feedback is provided, the device comprising a housing that includes a display on a front portion of the housing, and a recessed portion on a back portion of the housing. The device further includes a camera arm that is configured to be fully inserted in the recessed portion of the housing in a first configuration, and that is configured to be partially inserted in the recessed portion of the housing in a second configuration, wherein the camera arm includes a camera on one end of the camera arm that extends out from the side of the housing. The device further includes a hinge attached to the back portion of the housing that causes the housing the be placed at a first angle when in the first configuration, and that causes the housing to be placed at a second angle when in the second configuration.

In some embodiments, a method (which may be performed by a system comprising a display device and processing system) comprises receiving a sequence of camera images of a user performing an exercise, identifying a plurality of anatomical points of the user from the sequence of camera images, and determining user performance information for the exercise based on an analysis of the plurality of anatomical points. The method may further comprise applying exercise performance rules to the user performance information, the exercise performance rules including criteria for satisfactory performance of the exercise and providing corrective feedback to the user for exercise performance rules that are not met by the user performance information.

In some embodiments, the exercise comprises a plurality of movement stages to be performed in sequence and the step of determining the user performance information for the exercise further comprises analyzing the plurality of anatomical points to determine whether the user has performed the movement associated with each of the movement stages of the exercise. The method may further comprise displaying exercise content associated with the exercise, wherein the sequence of camera images is captured while the exercise content is being displayed.

In some embodiments, the method further comprises displaying content for the exercise on a display device comprising a housing having a recessed portion and a camera arm configured to be inserted in the recessed portion of the housing in a first position and partially inserted in the recessed portion of the housing in a second position, and extending the camera arm to expose a camera on one end thereof to position the camera to capture the sequence of camera images while the exercise content is being displayed.

In some embodiments, the sequence of camera images comprises a sequence of two-dimensional images and identifying a plurality of anatomical points of the user further comprises identifying the plurality of anatomical points on the two-dimensional images, and the method further comprises converting the two-dimensional anatomical points to three-dimensional anatomical points. The method may further comprise identifying performance criteria for the exercise, including an angle and/or position of at least one anatomic point during a movement stage of the exercise, and tracking the plurality of anatomical points in three-dimensional space to identify whether the performance criteria for the exercise is met. The method further comprises providing corrective feedback to the user if the performance criteria for the exercise is not met.

In some embodiments, the exercise has associated ground truth values represented as a sequence of anatomical angles and/or positions, and wherein the user performance information comprises comparison results between the ground truth values for the exercise and the identified plurality of anatomical points of the user. The method may further comprise calculating a score for the user's performance of the exercise based on comparison results between the ground truth values for the exercise and the identified plurality of anatomical points of the user. A user interface may be presented that includes a summary of the exercise including feedback associated with the user's performance of the exercise.

In some embodiments, exercise content is generated through a process comprising receiving exercise content from an exercise content provider, identifying a sequence of exercises within the exercise content and associated timing information for each exercise, identifying, for each exercise, at least one correction, and presenting a user interface to the exercise content provider for verification of the identified exercises, timing information, and at least one correction. The exercise content may be further generated through a process comprising receiving, from the exercise content provider, verification of identified exercises, timing information and at least one correction, receiving a user feedback message from the content provider for the at one correction, and storing the exercise content including identified exercises, associated timing information, identified corrections, and the user feedback message.

In some embodiments, the exercise is a mat-based exercise, a weightlifting exercise, a treadmill exercise, a cycling exercise, and/or a rowing exercise.

In various embodiments, a system comprises a display device configured to displaying exercise content, the display device comprising a housing, a display screen on a front portion of the housing and a recessed portion on a back portion of the housing, and a camera arm configured to be inserted in the recessed portion of the housing in a first position and partially inserted in the recessed portion of the housing in a second position. The camera is further configured to extend to expose the camera on one end thereof to capture the sequence of camera images of the user performing the exercise while the exercise content is being displayed.

In some embodiments, the display device is further configured to receive a sequence of images from the camera of a user performing an exercise, wherein the sequence of images comprises two-dimensional images, wherein the display device is further configured to identify a plurality of anatomical points of the user on the two-dimensional images, and wherein the method further comprises converting the two-dimensional anatomical points to three-dimensional anatomical points.

In some embodiments, the system further includes a mat, free weights, a weight machine, a treadmill, a stationary bike, and/or a rowing machine. The camera arm may comprise magnetic components and an electronic communications port, and wherein the camera arm is detachable from the housing and adapted to attach to the housing via a magnetic connection with a corresponding magnetic element of the housing; and wherein the electronic communications port is configured to align with a corresponding port of the housing for electronic communications with the display device. The display device may be configured to display a dashboard screen to the user after an exercise session, including exercise corrections and highlights of the user's performances during the exercise session.

The scope of the present disclosure is defined by the claims, which are incorporated into this section by reference. A more complete understanding of the present disclosure will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A and 1B show front perspective views of a device for presenting exercise content and detecting user performance information, in accordance with some embodiments of the disclosed subject matter.

FIG. 2 shows a front perspective view of a device for presenting exercise content and detecting user performance information, including an image capture component disposed on an arm, in accordance with some embodiments of the disclosed subject matter.

FIG. 3 shows a front perspective view of a device for presenting exercise content with a closed cover, in accordance with some embodiments of the disclosed subject matter.

FIG. 4 shows a rear perspective view of a device for presenting exercise content and detecting user performance information, in accordance with some embodiments of the disclosed subject matter.

FIG. 5 shows a rear perspective view of a back portion of a device for presenting exercise content and detecting user performance information, with a hinge of the device opened in accordance with some embodiments of the disclosed subject matter.

FIG. 6 shows a front view of a device for presenting exercise content and detecting user performance information, in accordance with some embodiments of the disclosed subject matter.

FIG. 7 shows a rear view of a device for presenting exercise content and detecting user performance information, in accordance with some embodiments of the disclosed subject matter.

FIG. 8 shows a top view of a device for presenting exercise content and detecting user performance information, in accordance with some embodiments of the disclosed subject matter.

FIG. 9 shows a side view of a device for presenting exercise content and detecting user performance information, in accordance with some embodiments of the disclosed subject matter.

FIG. 10 shows an illustrative example of a process for processing an exercise video uploaded from an instructor device, in accordance with some embodiments of the disclosed subject matter.

FIG. 11 shows an illustrative example of a process for presenting an exercise session in connection with feedback based on user information captured during the exercise session, in accordance with some embodiments of the disclosed subject matter.

FIG. 12 shows a schematic diagram of an illustrative system suitable for implementation of mechanisms described herein for presenting exercise content with feedback, in accordance with some embodiments of the disclosed subject matter.

FIG. 13 shows a detailed example of hardware that can be used in a server and/or a user device of FIG. 12, in accordance with some embodiments of the disclosed subject matter.

FIGS. 14A, 14B, and 14C show illustrative examples of user interfaces for presenting an exercise session and presenting workout feedback, in accordance with some embodiments of the disclosed subject matter.

FIG. 15 shows an illustrative example of a user interface for receiving information in connection with an uploaded exercise video from an instructor device, in accordance with some embodiments of the disclosed subject matter.

FIGS. 16-21 show rear views of a device for presenting exercise content and detecting user performance information during an exercise session with a power supply for the device in accordance with some embodiments of the disclosed subject matter.

FIGS. 22-24 show views of a stand for a device for presenting exercise content and detecting user performance information during an exercise session, in accordance with some embodiments of the disclosed subject matter.

FIGS. 25A, 25B, and 26 show views of a camera arm that includes a camera of a device for presenting exercise content and detecting user performance, in accordance with some embodiments of the disclosed subject matter.

FIGS. 27A and 27B show example views of a device for presenting exercise content and detecting user performance information in connection with a cover of the device, in accordance with some embodiments of the disclosed subject matter.

FIGS. 28A, 28B, and 28C show examples of an exercise mat in connection with a device for presenting exercise content and detecting user performance information, in accordance with some embodiments of the disclosed subject matter.

FIG. 29 shows an example of a power button for a device for presenting exercise content and detecting user performance information, in accordance with some embodiments of the disclosed subject matter.

FIG. 30 shows an example of a rear view of a device for presenting exercise content and receiving user performance information, in accordance with some embodiments of the disclosed subject matter.

FIGS. 31A and 31B show examples of a user exercising on a mat while using a device for presenting exercise content and detecting user performance information, in accordance with some embodiments of the disclosed subject matter.

FIGS. 32A and 32B show an example of a camera image and associated anatomical points, in accordance with some embodiments of the disclosed subject matter.

FIGS. 33A and 33B show examples of image and anatomical point processing, in accordance with some embodiments of the disclosed subject matter.

### DETAILED DESCRIPTION

In accordance with various embodiments, mechanisms (which can include methods, systems, and media) for presenting exercise content and providing exercise feedback are provided.

In some embodiments, the mechanisms described herein include a workout device that includes a display on which an exercise video can be presented. In some embodiments, the workout device can be attached to or placed on an exercise mat (e.g., a yoga mat) on which a user can perform one or more exercises included in the exercise video.

Additionally, in some embodiments, the workout device has a camera arm that includes a camera. In some embodiments, the camera is configured to capture images of the user while performing exercises during an exercise session, and machine vision is used to provide feedback to the user, such as corrections to the user's exercise form and tracking the number of repetitions completed of an exercise. In some embodiments, the camera images are sent to a server to identify feedback for the user during performance of the exercise. For example, in some embodiments, the camera images can be used to determine whether the user has correct/incorrect form during a particular exercise (e.g., that the feet of the user are not wide enough apart, that the user is not bending low enough during a particular exercise, etc.) and/or that the user isn't performing the exercise at an anticipated rate (e.g., if the user is slowing down during performance of a particular exercise). Continuing with this example, in some embodiments, the feedback can then be provided to the user during the exercise session and/or following completion of the exercise session (e.g., following completion of an exercise video).

In some embodiments, the camera images can be processed by the workout device, a mobile device, or other user device to identify the feedback to provide the user. For example, in some embodiments, the mechanisms described herein can identify anatomical points of a user included in an image (e.g., ankles, knees, hips, shoulders, etc.) and can identify the feedback based on the identified anatomical points of the user. As a more particular example, in some embodiments, the mechanisms can identify an exercise that the user is performing and can determine whether the user is performing the exercise correctly based on a positioning of the anatomical points of the user. In some embodiments, each exercise comprises one or more stages and the mechanisms track the anatomical points of the user to determine whether the user is progressing through each stage of the exercise.

In some embodiments, the workout device captures a series of two-dimensional images, identifies anatomical points on the body of the user, and converts the points to a three-dimensional representation of the user during exercise. The workout device, mobile system, user device and/or one or more server systems analyzes the three-dimensional representation to identify feedback in relation to the user's performance, which is delivered through video (e.g., video of the instructor providing verbal feedback, video of correct form), audio (e.g., audio of the instructor providing verbal feedback), and as displayed information (e.g., repetitions counted through the machine vision process).

In some embodiments, the mechanisms described herein can calculate a score for the user for the exercise video. In some embodiments, the score can be based on the feedback provided to the user for different exercises included in the video. For example, in an instance in which feedback is provided to the user that the user did not perform one or more exercises with correct form, the score can be relatively lower than in an instance in which the mechanisms determine that the user performed the exercises with correct form. In some embodiments, the feedback and/or the score corresponding to the exercise video can be saved in connection with a user account of the user such that the feedback and/or the score can be retrieved by the user at any suitable time. For example, in some embodiments, multiple scores for different exercise videos can be retrieved to show a historical trend of scores of the user over time.

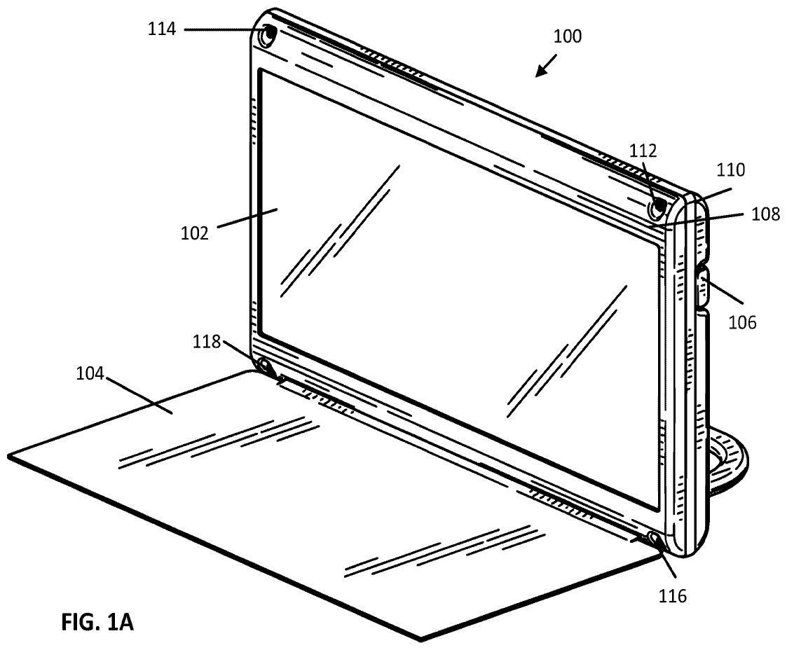

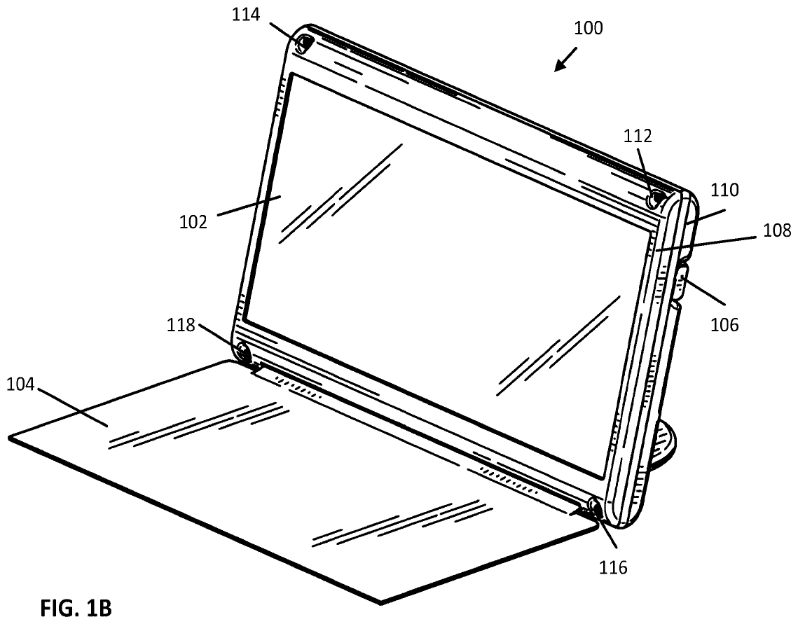

These and other features of exercise systems, methods, and media for presenting exercise content with feedback are described in connection with FIGS. 1A-15.

Turning to FIGS. 1A-B, an illustrative example is provided of a front perspective view of a device for presenting exercise content in connection with feedback for a user based on detected user performance information during an exercise session, in accordance with some embodiments of the disclosed subject matter. As illustrated, FIGS. 1A-B include a device 100, including a display 102, a cover 104, and a camera arm 106.

In some embodiments, display 102 can be any suitable screen on which an exercise content and/or feedback for a user can be presented. In some embodiments, display 102 can be any suitable type of screen or display, such as an LCD display, a touchscreen, a tablet computer, a laptop computer, and/or any other suitable type of screen or display. In some embodiments, display 102 can be within a housing that has a front portion 108 and a back portion 110. In some embodiments, front portion 108 and back portion 110 can be attached via any suitable screws, such as screws 112, 114, 116, and/or 118. In some embodiments, the display may be integrated with other components, including one or more speakers for playing audio to the user, one or more microphones for receiving audio (e.g., speech) from the user, user input/output controls (e.g., buttons, dials, touchscreen), one or more cameras, etc.

In some embodiments, the cover 104 can be any suitable cover which can be positioned to cover display 102. In some embodiments, cover 104 can be made of any suitable material or combination of materials (e.g., fabric, plastic, and/or any other suitable material). In some embodiments, cover 104 can be attached to an edge of front portion 108 of the display housing in any suitable manner (e.g., one or more screws, and/or in any other suitable manner).

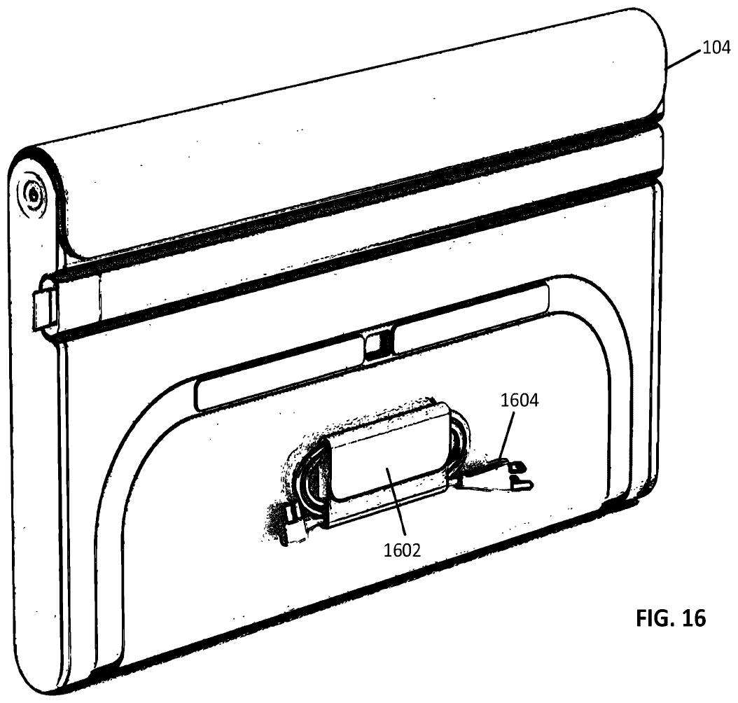

Additionally, note that, in some embodiments, cover 104 can be longer than a height of display 102. For example, as shown in FIG. 16, in some embodiments, cover 104 can be long enough such that an end portion of cover 104 wraps around a top of device 100 to attach to back portion 110. In some such embodiments, the end portion of cover 104 can include any suitable magnets such that the end portion of cover 104 magnetically attached to a top portion of back portion 110. In some embodiments, the magnets at the end portion of cover 104 can attach to a top portion of back portion 110 (e.g., to maintain cover 104 in a closed position that protects display 102 of device 100) and the same magnets at the end portion of cover 104 can also attach to an exercise mat, such as mat 208 (e.g., to orient the mat in a position relative to device 100). In some embodiments, the cover 104 is adapted to protect the display 102 when not in use, such as during storage or travel.

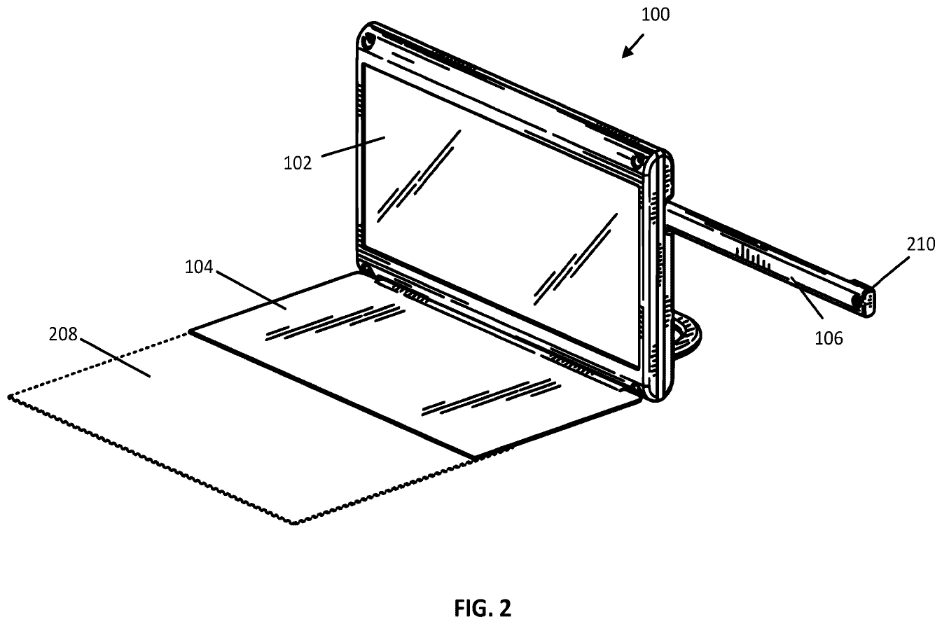

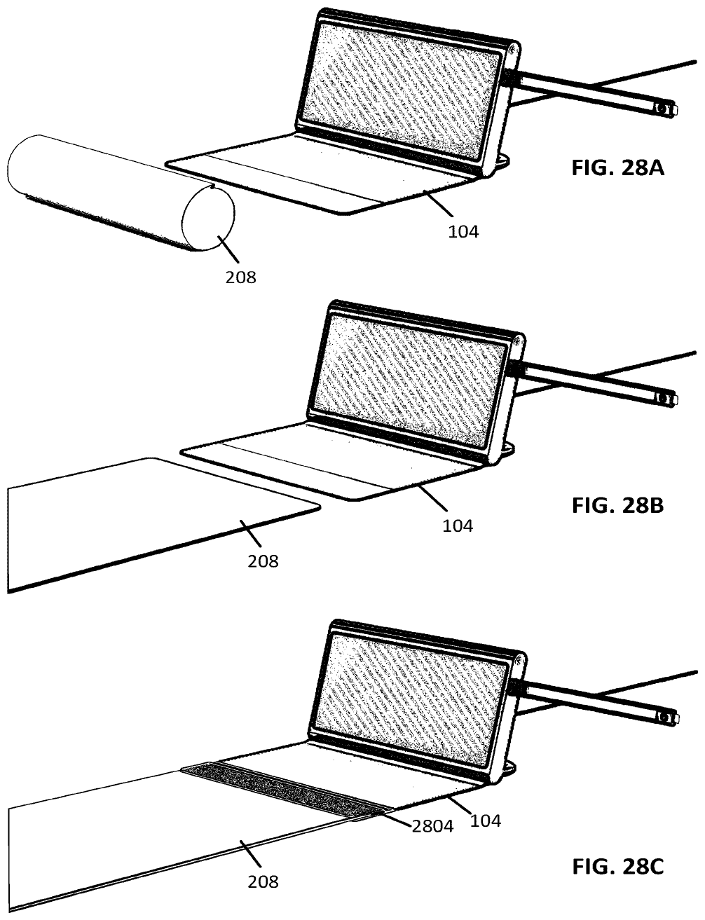

In some embodiments, the cover 104 has an open position such that the cover lies flat on a surface (e.g., as shown in FIGS. 2, 28A, 28B and 28C) and can be used to align and position the device 100, display 102 and camera 210 with respect to exercise equipment (e.g., a mat 208) and an exercise space.

Note that camera arm 106 is shown in a recessed position in FIG. 1A and FIG. 1B. In some embodiments, the recessed position can be used when device 100 is not in use and/or when transporting device 100. In some embodiments, camera arm 106 can be extended, as shown in and discussed below in connection with FIG. 2. Note that, in some embodiments, the cover 104 may extend over the rear of the device 100, and external features of the housing, including the recessed portion of device 100 for receiving the camera arm 106 and the stand 402, may be integrated into the cover 104 in some embodiments.

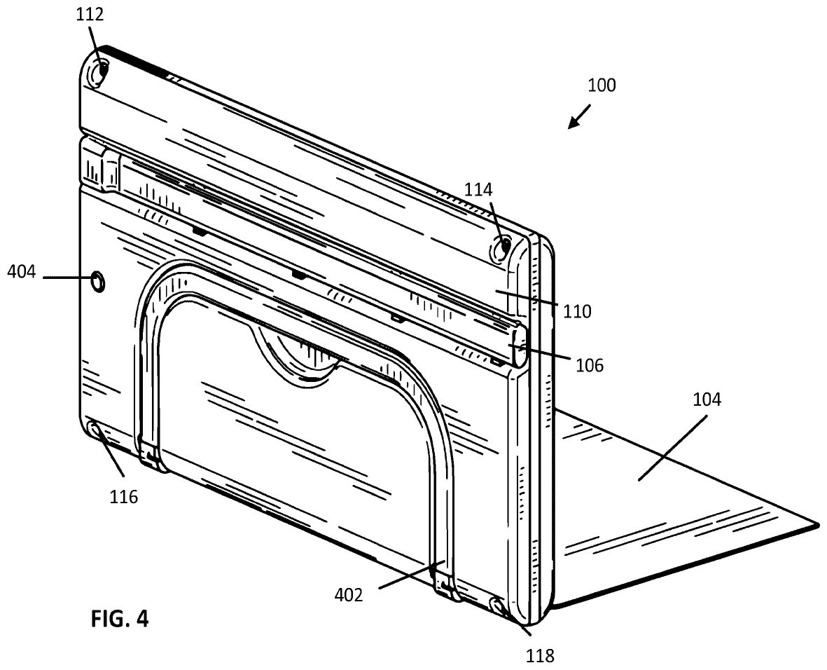

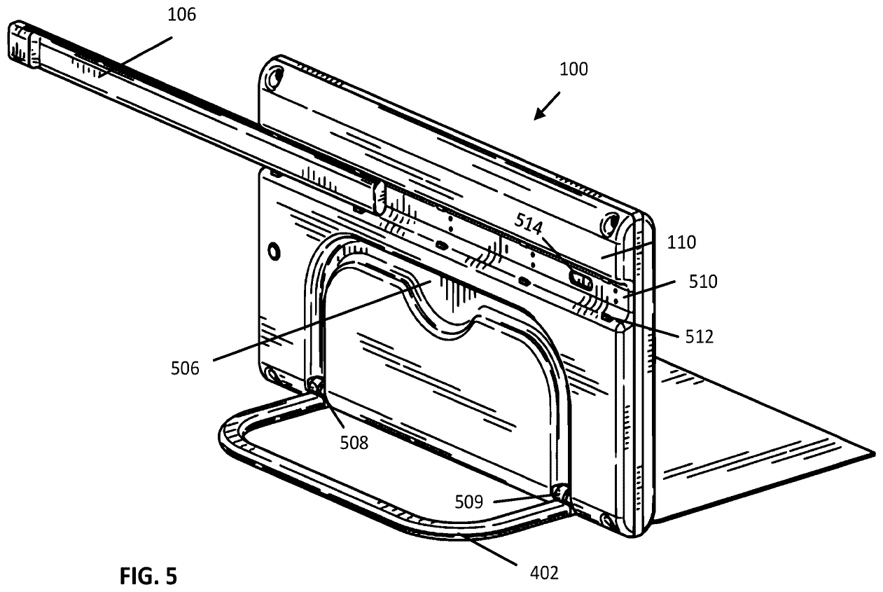

Note that, in some embodiments, device 100 can be positioned at any suitable angle for viewing by a user during exercise. For example, as shown in FIG. 1A, in some embodiments, device 100 can be positioned such that display 102 is at a 90-degree angle with cover 104. Turning to FIG. 1B, an illustrative example of a front perspective view of device 100 positioned with a different angle between display 102 and cover 104 is shown in accordance with some embodiments of the disclosed subject matter. As illustrated, device 100 can be positioned such that an angle between display 102 and cover 104 is more than 90 degrees (e.g., 100 degrees, 135 degrees, and/or any other suitable angle). In some embodiments, display 102 can be positioned via a stand located at a back portion of device 100, as shown in and described below in more detail in connection with FIGS. 4 and 5. In some embodiments, the display includes a pivoting display mount providing the display with 360-degree rotation for any viewing angle.

Turning to FIG. 2, an illustrative example of device 100 in which camera arm 106 of device 100 is extended is shown in accordance with some embodiments of the disclosed subject matter.

As illustrated in FIG. 2, in some embodiments, camera arm 106 can be extended or pulled out from the recessed position shown in FIGS. 1A-B. In some embodiments, extending camera arm 106 can cause camera 210 to be exposed, as shown in FIG. 2. In some embodiments, camera 210 can be any suitable camera for capturing videos and/or images of a user during presentation of exercise content on display 102. For example, in some embodiments, device 100 can be attached to a mat 208 as shown in FIG. 2, and camera 210 can be angled in the extended position such that camera 210 captures a perspective view of mat 208, and therefore, of a user exercising on the mat 208.

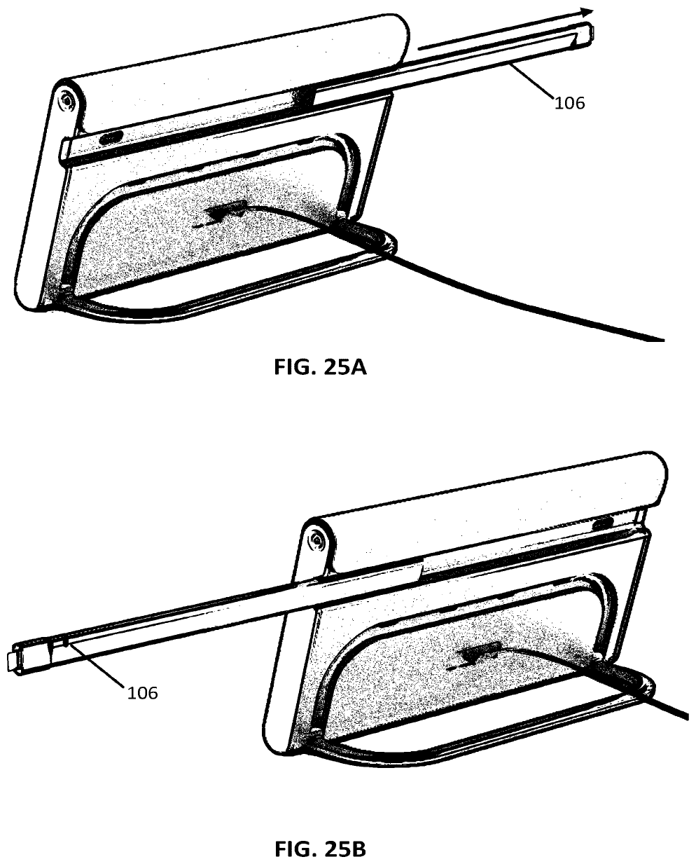

Note that, although camera 210 is shown on a right side of camera arm 106, in some embodiments, camera 210 can be located on a left side of camera arm 106. Additionally, note that, in some embodiments, camera arm 106 can be placed in the extended position in any suitable manner. For example, in some embodiments, camera arm can be removed from a recessed portion of device 100 (shown in and described below in connection with FIG. 5), positioned in the extended position, and pushed back in such a manner that only a portion of camera arm 106 is in the recessed portion (as shown in FIG. 5). Additionally, note that, as shown in FIGS. 25A and 25B, camera arm 106 can be positioned on a right side or a left side of device 100. Note further that, although a single camera is shown, in various embodiments, one or more additional cameras may be included in the housing or in a second extendable camera arm (e.g., extended to the opposite side of the housing as camera arm 106.

Additionally, note that, in some embodiments, camera arm 106 can have any suitable magnetic and/or electrical connections with device 100 such that data captured from camera 210 can be transmitted to device 100 and such that camera 210 can receive instructions from device 100. For example, the recessed portion of device 100 can include corresponding magnetic and/or electrical connections that mate with the magnetic and/or electrical connections on camera arm 106 (e.g., one connector for the placing camera arm 106 in an extended position on the left side of device 100 and another connector for placing camera arm 106 in an extended position on the right side of device 100). In another example, connecting the magnetic and/or electrical connections on camera arm 106 with the corresponding magnetic and/or electrical connections in the recessed portion of device 100 can cause camera arm 106 to be held in a fixed position in the recessed portion of device 100. In yet another example, connecting the magnetic and/or electrical connections on camera arm 106 with the corresponding magnetic and/or electrical connections in the recessed portion of device 100 can cause data (e.g., video data, audio data, etc.) from camera 210 to be transmitted to a suitable processing device within device 100.

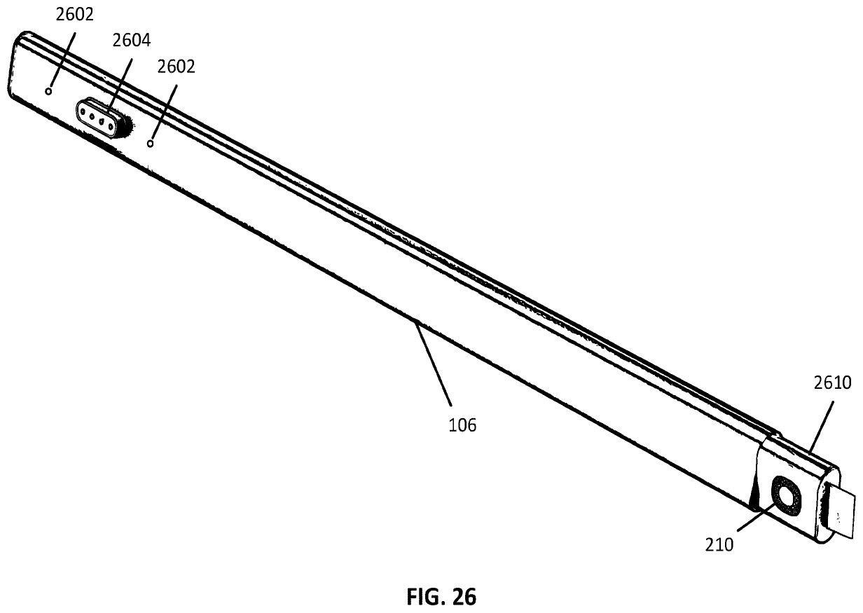

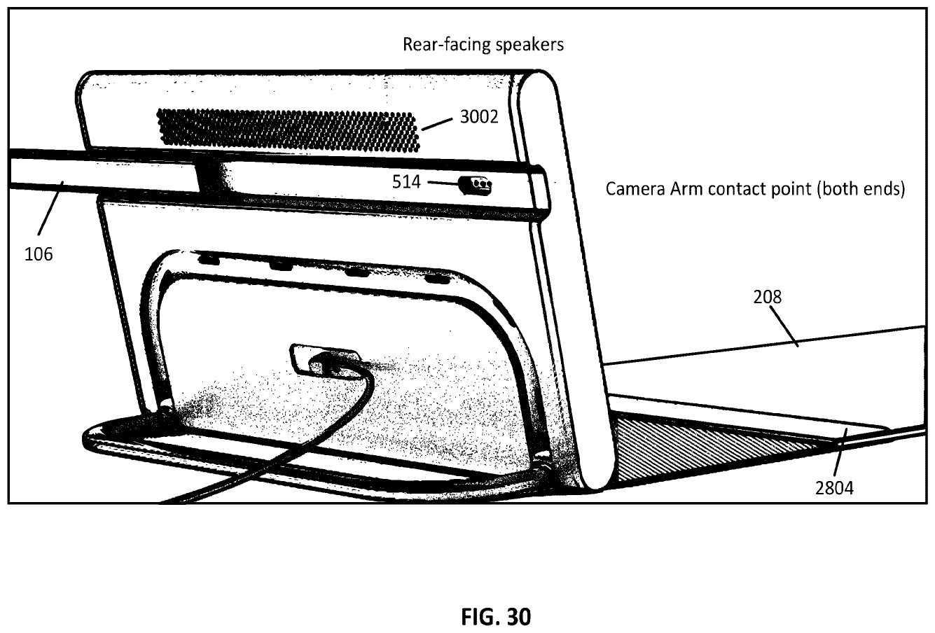

Turning to FIG. 26, an example view of a camera arm 106 is shown in accordance with some embodiments of the disclosed subject matter. As illustrated, in some embodiments, camera arm 106 can include any suitable magnets 2602 that allow camera arm 106 to be coupled to device 100. Additionally, in some embodiments, camera arm 106 can include contact points 2604 that correspond to any suitable magnetic and/or electrical connections (e.g., connector 514 as shown in FIGS. 5 and 30) in a back portion of device 100. In some embodiments, camera 210 of camera arm 106 can be on a camera portion 2610 that is at an end of camera arm 106. In some embodiments, camera portion 2610 can rotate in any suitable manner such that an angle of camera 210 can be adjusted upward or downward. The camera 210 may be any camera configured to capture image frames of a view of view including an exercise area and may include components such as memory components, processing components, audio capture components, communications components, optical components, and other camera components.

Note that, in some embodiments, device 100 can be attached to mat 208 in any suitable manner, such as magnets, snaps, clasps, hooks, Velcro®, etc. For example, in some embodiments, device 100 can be attached to mat 208 using one or more magnetic clips that attach device 100 to mat 208. In some such embodiments, a magnetic clip can be shaped like a clasp to attach device 100 to mat 208. In some embodiments, the magnetic clips can be located at any suitable position on device 100 and/or mat 208, such as on an underside of cover 104, and edge of device 100, a top portion of mat 208, and/or any other suitable position. Additionally, or alternatively, cover 104 can include a mechanical clip that holds mat 208 of any suitable length in a particular position in alignment with device 100. Additionally, or alternatively, cover 104 can include a portion that is configured to accommodate mat 208 of a particular thickness being placed beneath the portion of cover 104, as shown in and described below in connection with FIGS. 28A, 28B, and 28C. In some embodiments, the mat 208 identifies an exercise area allowing the display to be properly positioned to present exercise content to the user during exercise and the camera 210 to be properly aligned to capture images of the user during exercise for use in providing feedback as described herein.

Turning to FIGS. 28A, 28B, and 28C, example views of mat 208 connecting to a device via cover 104 are shown in accordance with some embodiments of the disclosed subject matter. As illustrated, mat 208 can be unrolled such that a first end of mat 208 connects to cover 104. As shown in FIG. 28C, mat 208 can be connected to cover 104 at an end portion 2804. In particular, in some embodiments, end portion 2804 can be opened such that the first end of mat 208 can be inserted in end portion 2804 and clasped in position to prevent slipping via magnets in end portion 2804. Note that another view of end portion 2804 holding mat 208 is shown in FIG. 30.

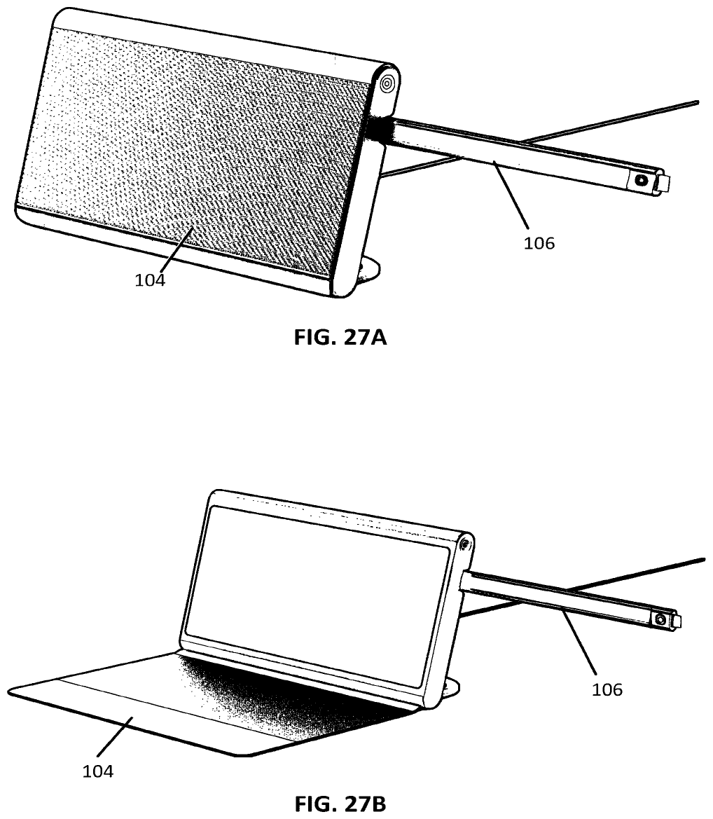

Turning to FIGS. 27A and 27B, additional views of device 100 with camera arm 106 extended are shown in accordance with some embodiments of the disclosed subject matter.

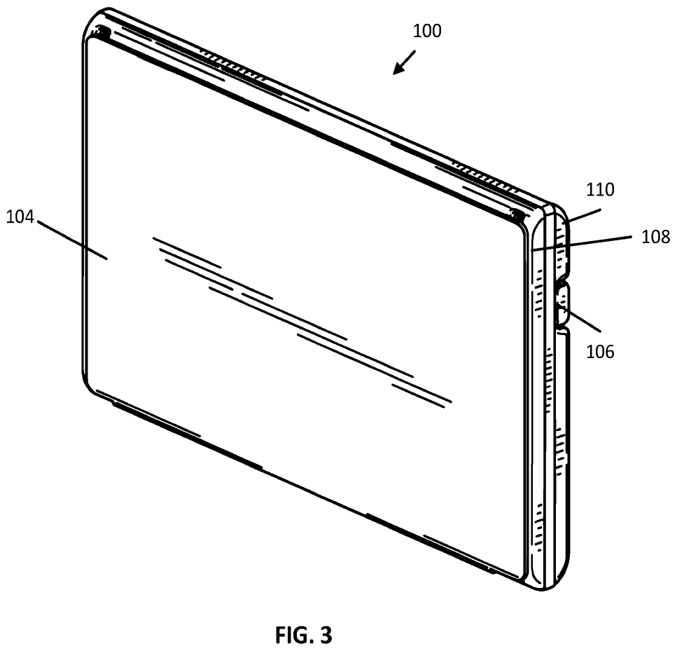

Turning to FIG. 3, an example front perspective view of device 100 with cover 104 in a closed position is shown in accordance with some embodiments of the disclosed subject matter. Note that, as shown in FIG. 3, camera arm 106 is shown in a recessed position. For example, device 100 can be placed into the closed position configuration shown in FIG. 3 for storage and/or transport.

Turning to FIG. 4, a rear perspective view of device 100 is shown in accordance with some embodiments of the disclosed subject matter. As illustrated, in some embodiments, the back portion of device 100 can include a stand 402 and/or a power button 404.

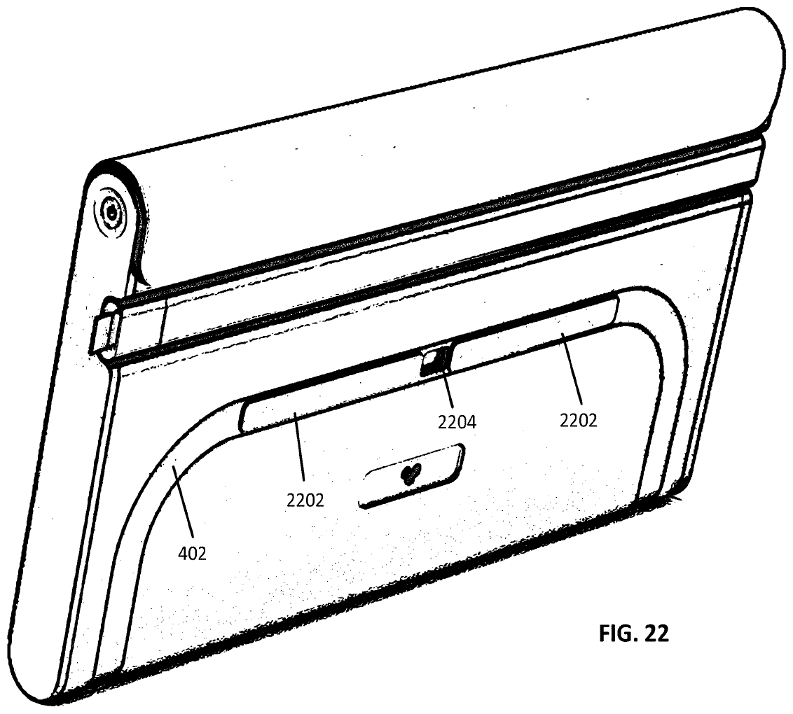

In some embodiments, stand 402 can be any suitable stand that can be used to prop up device 100 when device 100 is in use. In some embodiments, stand 402 can be folded into a recessed portion in back portion 110 of device 100 when stand 402 is not in use, as shown in FIG. 4. Note that stand 402 is shown in and described below in more detail in connection with FIGS. 22-24.

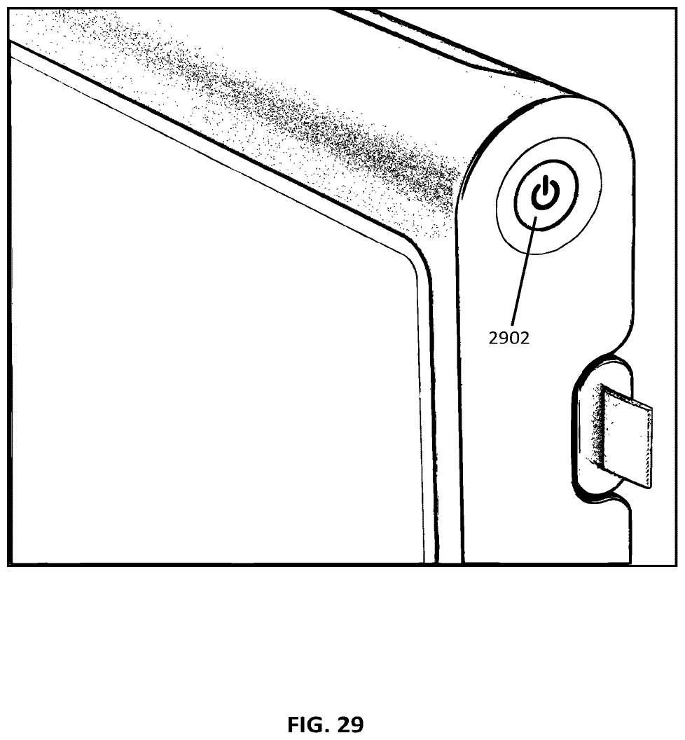

In some embodiments, power button 404 can be any suitable button that can be used to turn on device 100. Note that the location of power button 404 on device 100 is merely shown as an example, and, in some embodiments, power button 404 can be located at any suitable position on device 100 (e.g., on a top portion of device 100, on front portion 108 of device 100, a side of device 100, and/or at any other suitable position). For example, turning to FIG. 29, an example of device 100 with a power button 2902 positioned on a side of the device is shown in accordance with some embodiments of the disclosed subject matter.

Turning to FIG. 5, a rear perspective view of device 100 in which camera arm 106 is in an extended position is shown in accordance with some embodiments of the disclosed subject matter.

As illustrated, back portion 110 of device 100 can include a recessed portion 510 in which camera arm 106 can fit. Note that, in some embodiments, when camera arm 106 is in an extended position (as shown in FIG. 5), a portion of camera arm 106 can extend past recessed portion 510, and a reminder of camera arm 106 can be positioned within recessed portion 510. Additionally, note that, in some embodiments, recessed portion 510 can include one or more protrusions, such as protrusion 512. In some embodiments, the protrusions can be used to grip or otherwise support camera arm 106 within recessed portion 510. In some embodiments, as shown in FIG. 5, the protrusions can be located on a top edge and a bottom edge of recessed portion 510.

In some embodiments, recessed portion 510 can include connector 514 that mates with a corresponding connector on camera arm 106. For example, connector 514 and the corresponding connector on camera arm 106 can be a magnetic and electrical connection that magnetically supports camera arm 106 at a particular position within recessed portion 510 and that electrically connects the camera of camera arm 106 with device 100. In continuing this example, data captured by the camera of camera arm 106, which can include video data, audio data, focusing data, acceleration data of a subject, etc., can be transmitted from the camera of camera arm 106 to a suitable processor within device 100. Additionally, in some embodiments, power from device 100 can be provided to the camera on camera arm 106 via connector 514 and the corresponding connector on camera arm 106. Note that an additional view of connector 514 is shown in and described in connection with FIG. 30.

In some embodiments, connector 514 can assist a user of device 100 in positioning the camera on camera arm 106. For example, as shown in FIG. 5, recessed portion 510 can include connector 514 such that, upon mating connector 514 and a corresponding connector on camera arm 106, camera arm 106 can be supported in a fixed position in the recessed portion of device 100 in which the camera is extended on a left side of device 100 and at a particular distance from the body of device 100.

It should be noted that recessed portion 510 can include any suitable number of connectors. For example, recessed portion 510 can include a first connector for placing camera arm 106 in an extended position on the left side of device 100 and a second connector for placing camera arm 106 in an extended position on the right side of device 100. In another example, recessed portion 510 can include multiple connectors for placing camera arm 106 in an extended position and at different distances from the body of device 100.

Additionally, FIG. 5 shows stand 402 in use. In some embodiments, when stand 402 is in use, stand 402 can be rotated down from a recessed portion 506 in which stand 402 is stored when stand 402 is not in use. In some embodiments, stand 402 can then be placed on the ground such that stand 402 supports display 102 (not shown in FIG. 5) such that display 102 is provided in an upright position. Note that an angle between stand 402 and recessed portion 506 can be any suitable angle, such as 30 degrees, 45 degrees, 60 degrees, 90 degrees, and/or any other suitable angle. Note also that, in some embodiments, stand 402 can be configured such that stand 402 can have a rotational limit and/or can have predetermined rotational positions. For example, in some embodiments, stand 402 can be configured such that stand 402 can be rotated to an angle such that device 100 is at a 45-degree angle when in use. A more particular example is shown, for example, in FIG. 1B. In another example, in some embodiments, stand 402 can be configured such that stand 402 can be rotated to preset angles—e.g., a first rotational position of stand 402 causes the display to be placed at a 45-degree angle and a second rotational position of stand 402 causes the display to be placed at a 90-degree angle. More particular examples of varying rotational positions are shown, for example, in FIGS. 1A and 1B. Additionally, note that, in some embodiments, stand 402 can rotate downward from recessed portion 506 in any suitable manner. For example, in some embodiments, stand 402 can rotate using hinges 508 and 509, as shown in FIG. 5.

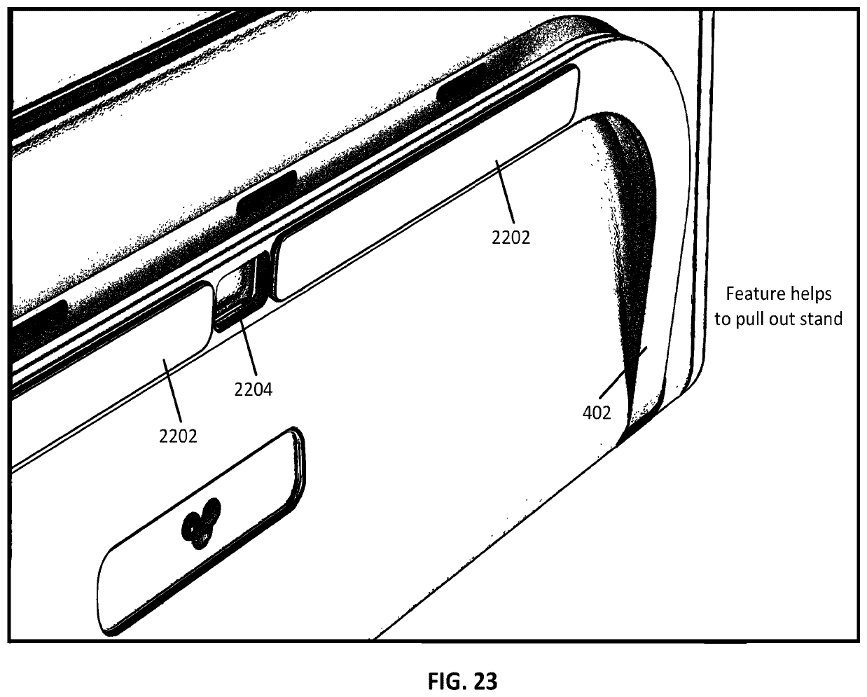

Turning to FIGS. 22-24, additional views of stand 402 are shown in accordance with some embodiments of the disclosed subject matter. As illustrated in FIGS. 22 and 23, in some embodiments, stand 402 can include any suitable grips 2202. In some embodiments, grips 2202 can be made of any suitable material or combination of materials (rubber, plastic, fabric, and/or any other suitable material) that allow stand 402, when rotated down from recessed portion 506, to be more securely placed on the ground. Additionally, in some embodiments, stand 402 can include an opening 2204. In some embodiments, opening 2204 can aid in allowing a user to grip stand 402 when rotating stand 402. For example, in some embodiments, opening 2204 can have a size or dimensions that correspond to a finger of a user.

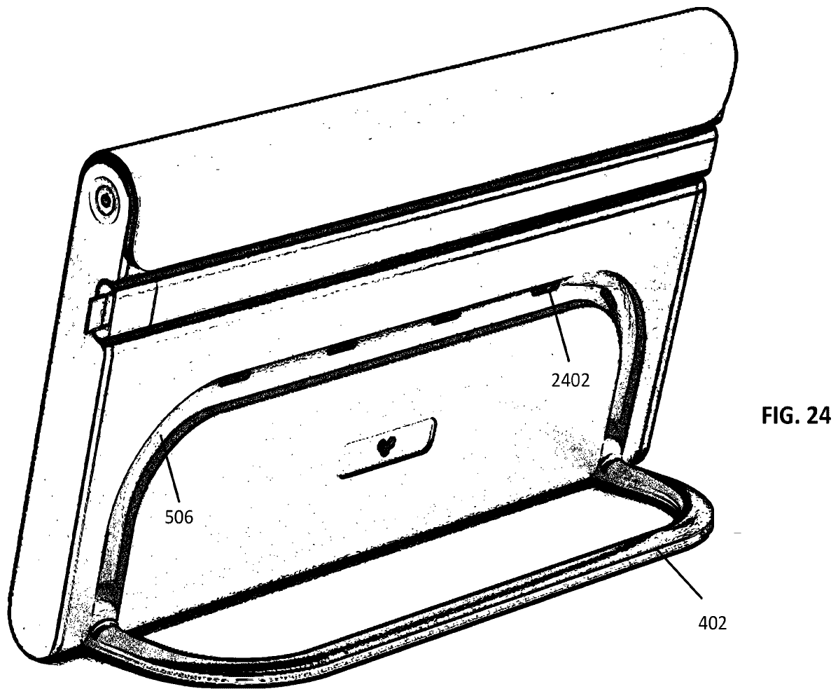

Turning to FIG. 24, an additional rear perspective view of device 100 when stand 402 is in a downward position is shown in accordance with some embodiments of the disclosed subject matter. As illustrated, in some embodiments, recessed portion 506 can have any suitable vents 2402. In some embodiments, vents 2402 can allow for cooling of device 100 when in use.

Turning to FIG. 6, a front view of device 100 in which camera arm 106 is in an extended position on the right side of device 100 is shown in accordance with some embodiments of the disclosed subject matter.

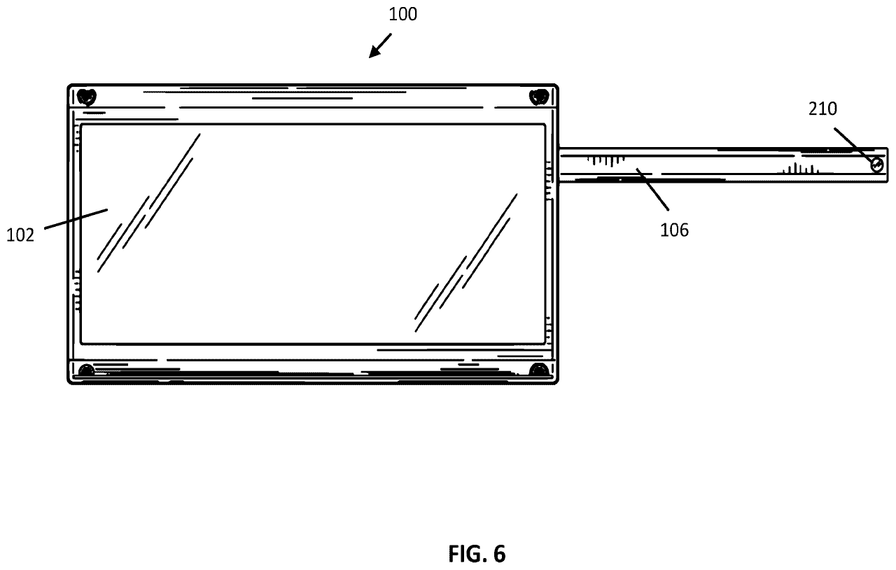

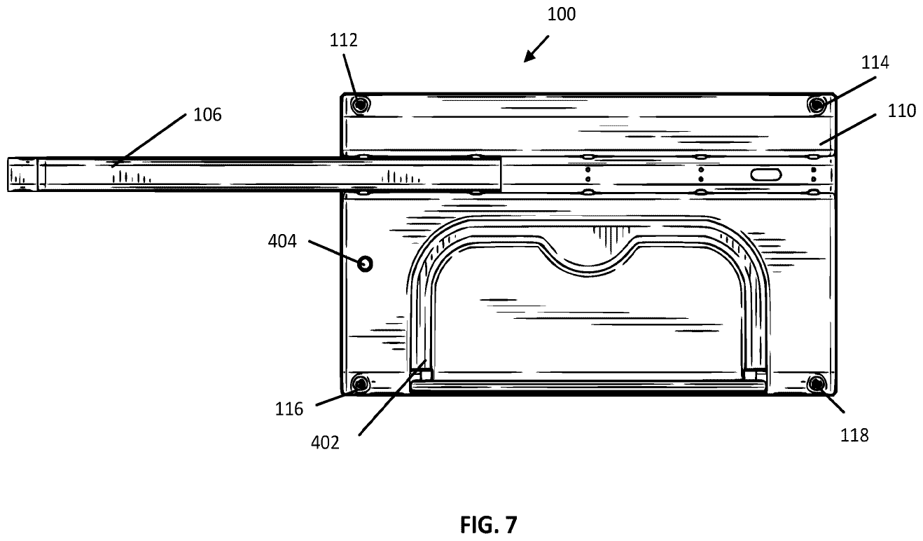

Turning to FIG. 7, a rear view of device 100 in which camera arm 106 is in an extended position on the right side of device 100 is shown in accordance with some embodiments of the disclosed subject matter.

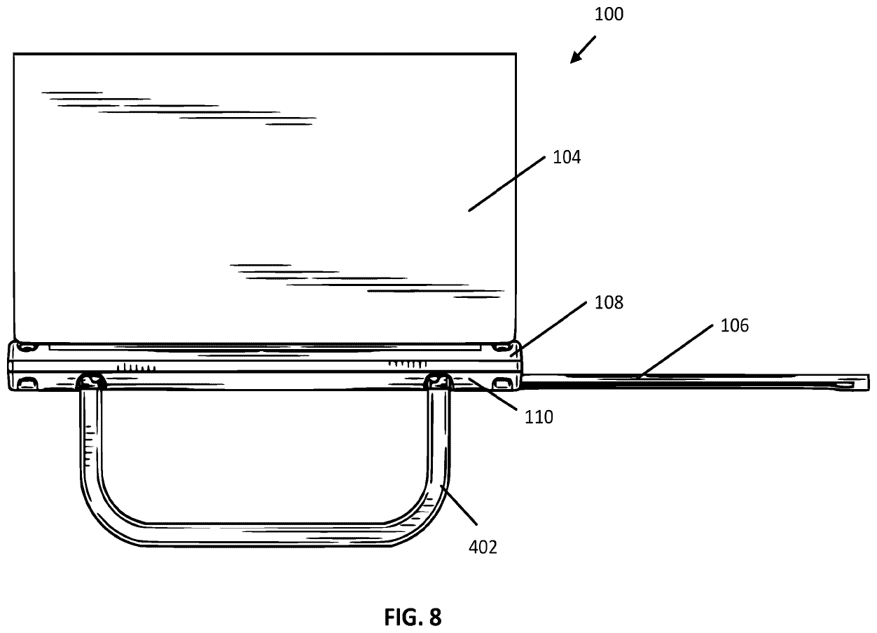

Turning to FIG. 8, a top view of device 100 in which camera arm 106 is in an extended position and in which stand 402 is in an extended position is shown in accordance with some embodiments of the disclosed subject matter.

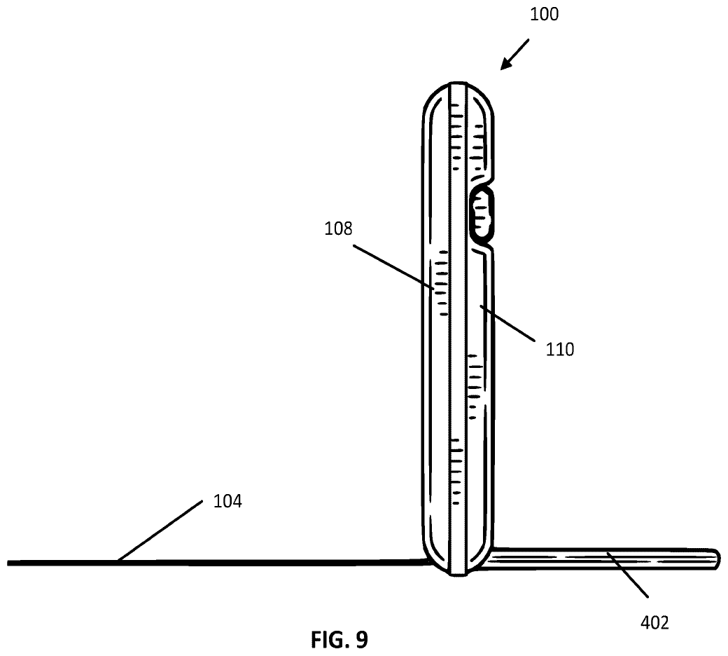

Turning to FIG. 9, a side view of device 100 in which camera arm 106 is in a recessed position and in which stand 402 is in an extended position is shown in accordance with some embodiments of the disclosed subject matter.

Note that, in some embodiments, device 100 can have any suitable power supply. For example, in some embodiments, device 100 can use any suitable battery. As another example, in some embodiments, device 100 can have a plug-in power supply that has a port at any suitable position on device 100.

Turning to FIGS. 16-21, example rear views of device 100 in connection with a plug-in power supply are shown in accordance with some embodiments of the disclosed subject matter.

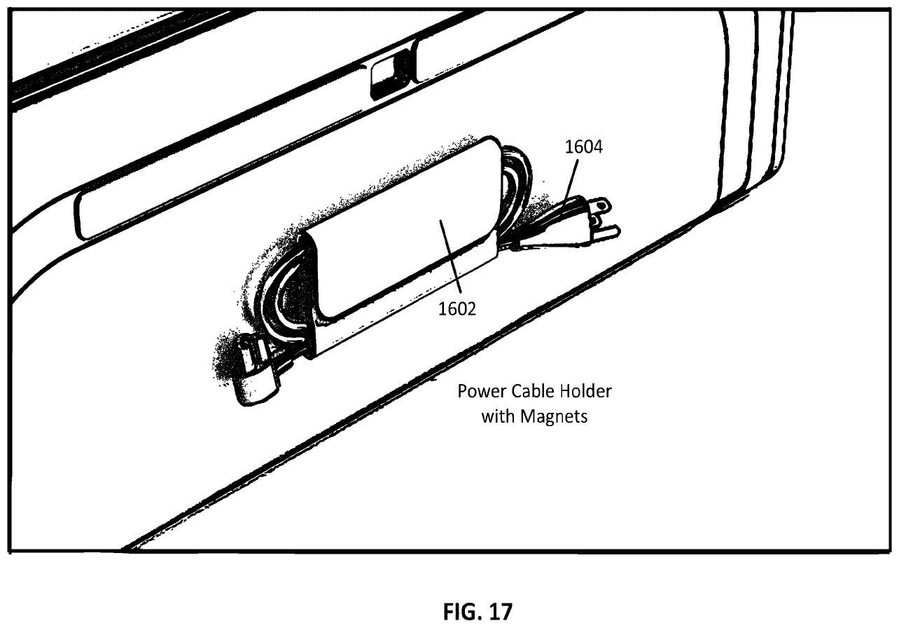

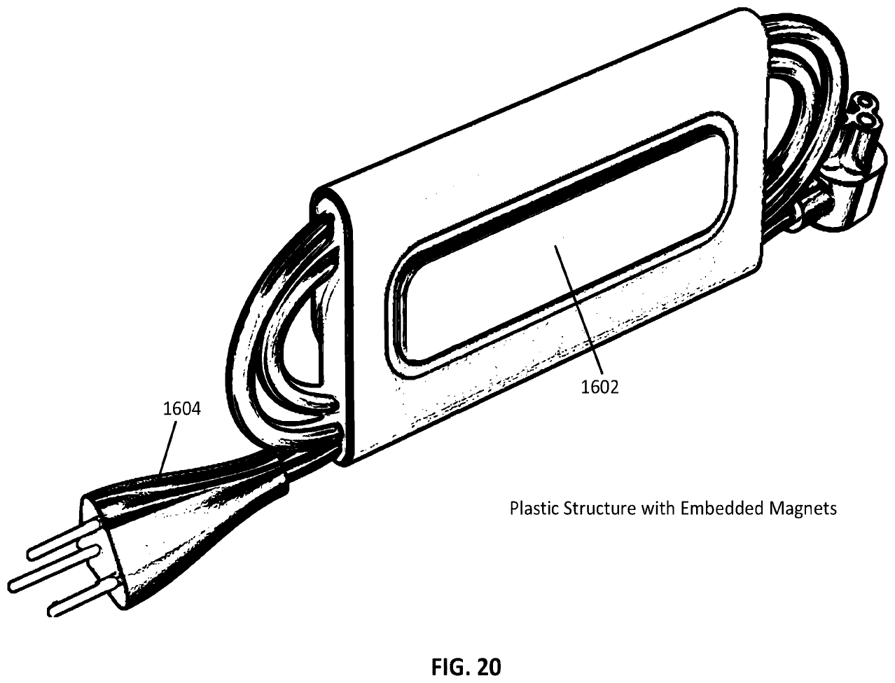

As illustrated in FIG. 16, device 100 can include a power cord 1604 that can be plugged into any suitable outlet or wall power supply. In some embodiments, power cord 1604 can be held by a power cable holder 1602. In some embodiments, power cable holder 1602 can attach to a back portion of device 100 via one or more magnets in power cable holder 1602 and/or one or more magnets in the back portion of device 100. An additional view of power cable holder 1602 and power cord 1604 is shown in FIG. 17. FIG. 20 shows another view of power cable holder 1602 and power cord 1604.

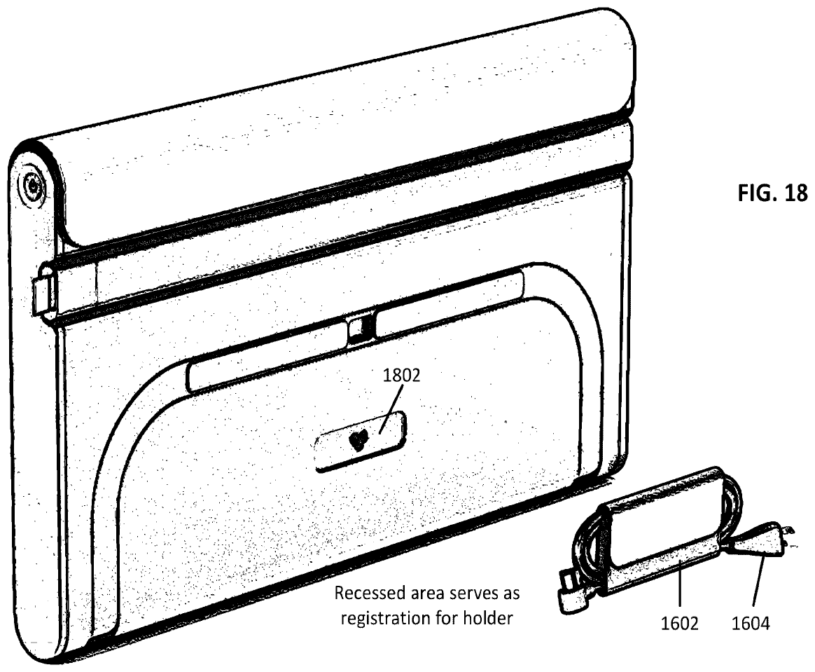

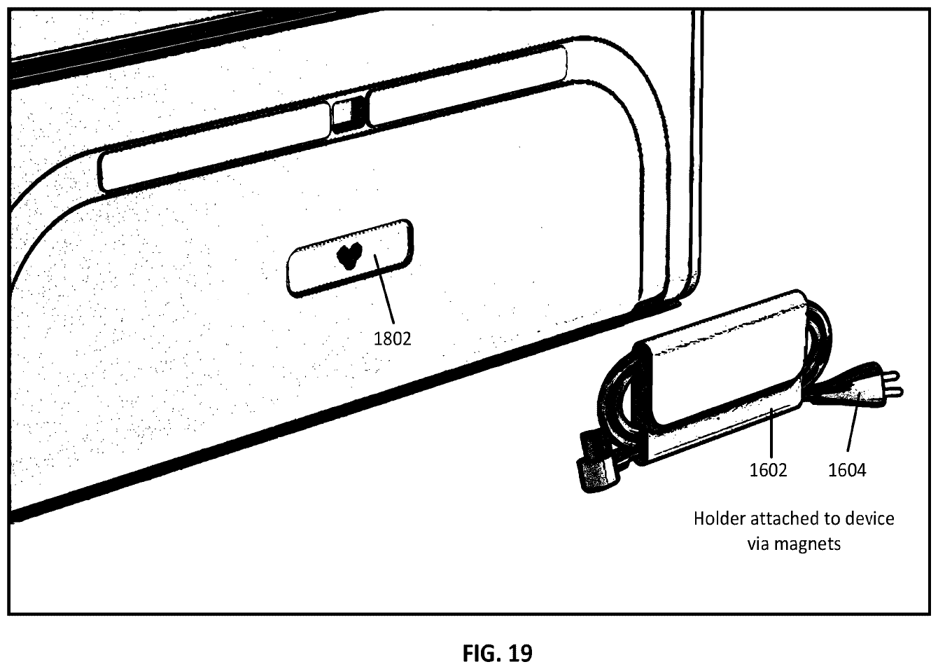

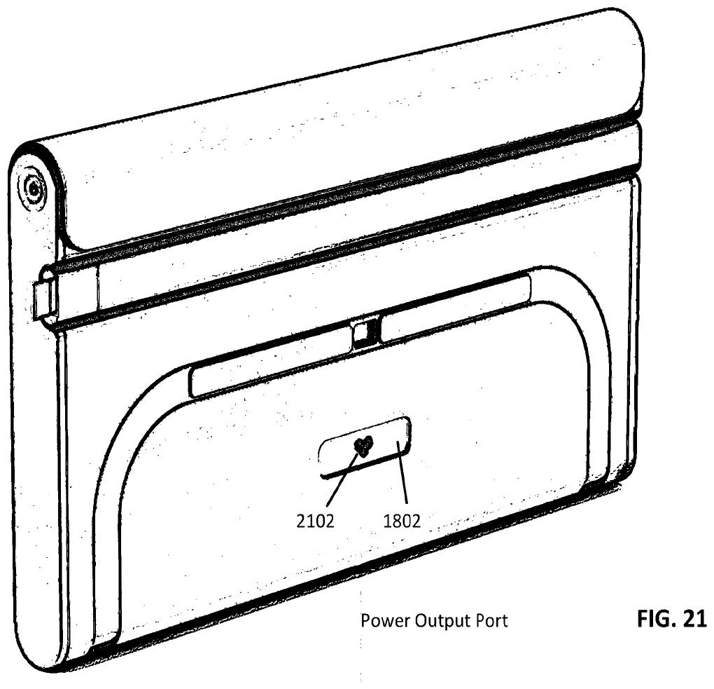

Turning to FIG. 18, an example of power cable holder 1602 and power cord 1604 removed from the back portion of device 100 is shown in accordance with some embodiments of the disclosed subject matter. As illustrated, in some embodiments, the back portion of device 100 can include a recessed area 1802 that is a holder for power cable holder 1602 when attached to device 100. FIG. 19 shows another view of recessed area 1802. Referring to FIG. 21, in some embodiments, recessed area 1802 can include a power output port 2102 to which an end of power cord 1604 can be plugged in.

In some embodiments, device 100 can include a speaker in any suitable location. For example, as shown in FIG. 30, in some embodiments, a speaker 3001 can be located on a back portion of device 100. The device 100 can also include a microphone in any suitable location.

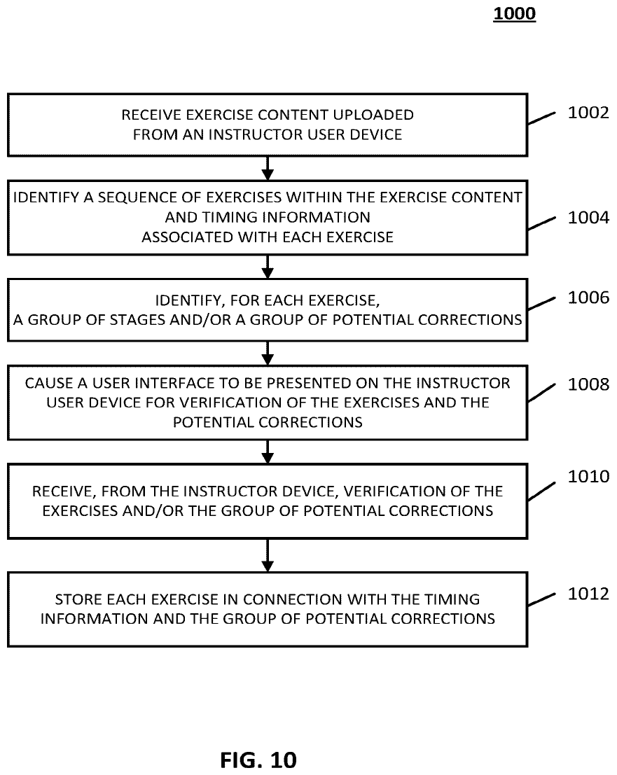

Turning to FIG. 10, an illustrative example 1000 of a process for receiving and annotating exercise content uploaded by an instructor is shown in accordance with some embodiments of the disclosed subject matter. In some embodiments, blocks of process 1000 can be executed by any suitable device, such as a server associated with an exercise video service that provides exercise videos to users, an application executed by a local mobile device (e.g., a tablet, laptop computer, smart phone, etc.), the workout device, etc.

Process 1000 can begin at 1002 by receiving exercise content (e.g., an exercise video) uploaded from an instructor user device. In some embodiments, process 1000 can receive a video from the instructor user device in any suitable manner. For example, in some embodiments, the video can be uploaded from the instructor user device to the server executing process 1000. Note that, in some embodiments, the video can be of any suitable duration (e.g., ten minutes, twenty minutes, thirty minutes, one hour, and/or any other suitable duration). Additionally, note that, in some embodiments, the video can include any suitable audio content (e.g., background music, spoken content from an instructor associated with the instructor user device, and/or any other suitable audio content). In some embodiments, the video represents an exercise session and may include video and audio of the instructor leading the exercise session, video of a person performing the exercises using proper form, video of an exercise class, or other video content. In some embodiments, the exercise content uploaded to the server may include still images, animations, screen messages, and other exercise content.

At 1004, process 1000 identifies a sequence of exercises within the video and timing information associated with each exercise. In some embodiments, the sequence of exercises within the video can correspond to any suitable exercises instructed by an instructor of the instructor device. For example, the sequence of exercises can correspond to a series of exercises to be performed in a workout routine instructed by the instructor. As a more particular example, the sequence of exercises can include any suitable exercises, such as a squat, a lunge, jumping jacks, yoga poses (e.g., chair pose, tree pose, etc.), and/or any other suitable exercises. In some embodiments, in an instance in which an exercise in the sequence of exercises is performed on each side of a user's body (e.g., a lunge with the right leg in front and a lunge with the left leg in front), the sequence of exercises can indicate that the particular exercise is to be performed twice, once on each side of the user's body. In some embodiments, in an instance in which a particular exercise is to be performed multiple times (e.g., a series of jumping jacks, a series of squats, etc.), the sequence of exercises can indicate a number of repetitions to be performed for the particular exercise (e.g., ten, fifteen, and/or any other suitable number of repetitions) and/or a time duration the exercise is to be repetitively performed for (e.g., twenty seconds, thirty seconds, one minute, and/or any other suitable duration).

In some embodiments, process 1000 can identify timing information associated with each exercise that indicates a time point or a time range during which each exercise is instructed within the video. For example, in an instance in which the sequence of exercises identified by process 1000 is: {squat; left leg lunge; right leg lunge}, the timing information can be: {1:05-1:15; 1:20-1:35; 1:40-1:55}, indicating that a squat (or a series of squats) is to be performed during 1:05-1:15 of the video.

In some embodiments, process 1000 can identify the sequence of exercises within the video and the timing information using any suitable technique or combination of techniques. For example, in some embodiments, process 1000 can analyze image data associated with one or more frames of the video to identify a particular exercise being performed by a person (e.g., the instructor, a person demonstrating the exercises, and/or any other suitable person) in the video. As a more particular example, in some embodiments, process 1000 can use any suitable image recognition techniques to identify any suitable features of the person in the video to identify the exercise the person in the video is performing. As a specific example, in some embodiments, process 1000 can identify any suitable anatomical points of a body of the person (e.g., ankles, knees, hands, shoulders, forehead, and/or any other suitable anatomical points) and can identify an exercise being performed by the person based on locations of the anatomical points and/or based on relative locations of the anatomical points. Continuing with this specific example, in an instance in which process 1000 identifies a series of anatomical points such as: 1) both feet of the user on the ground; 2) both knees bent at similar angles; and 3) arms of the user pointing up, process 1000 can determine that the person is likely to be performing chair pose, and/or any other suitable exercise that entails a person in a position matching the anatomical points.

In some embodiments, process 1000 can map anatomical points of a person in the video to a particular exercise in any suitable manner. For example, in some embodiments, process 1000 can use any suitable classification algorithm to determine whether the identified anatomical points correspond to exercises in a group of exercises. As a more particular example, in an instance in which the group of exercises includes a squat, a lunge, and chair pose, process 1000 can use any suitable type of trained classifier to determine whether the anatomical points correspond to each exercise of the group of exercise. In some such embodiments, a trained classifier used to identify a particular exercise can be trained in any suitable manner, such as using images or sequences of images that have been manually annotated to indicate the particular exercise corresponding to the image or the sequence of images as a training dataset. The training dataset can be used to train a neural network (e.g., a convolutional neural network) as an image classifier to identify exercises from the received video images.

In some embodiments, the server is trained to identify anatomical points from a video through a supervised machine learning process (e.g., trained convolutional neural network). In some embodiments, this process includes one or more neural networks trained using a dataset of annotated training images, which may include video images of people performing exercises that are annotated to identify desired anatomical points (e.g., hands, feet, elbows, shoulders, head, etc.). In various embodiments, any appropriate anatomical point detection algorithm may be used, such as active appearance model algorithms, active shape model algorithms, and other appropriate algorithms. In some embodiments, the location of the anatomical points on an image frame are mapped in three-dimensional space, allowing the system to track the user's posture and orientation, joint angles, distance moved, motion of body parts during exercise, relative position of body parts, etc. In some embodiments, the relative positions of anatomical points may be used to identify a pose or exercise (e.g., using trained neural network, using a rules-based classifier, etc.).

Note that, in some embodiments, process 1000 can use any other suitable information associated with the video to identify the exercises of the sequence of exercises. For example, in some embodiments, process 1000 can use spoken words included in the audio content to identify a particular exercise and/or to verify that an exercise identified based on a visual identification of anatomical points of a person in the video is correct. As a more particular example, in an instance in which process 1000 identifies a particular exercise as chair pose based on anatomical points of a person in a series of frames of the video, process 1000 can verify the identified exercise by determining whether the words “chair pose” are included in audio content associated with the series of frames. In some embodiments, an appropriate speech recognition algorithm is used (e.g., speech recognition methods based on trained neural networks, Hidden Markov Models, etc.) to identify speech from the corresponding audio and compare against known keywords associated with one or more exercises.

In some embodiments, the instructor or other individual manually annotates the video with information identifying the sequence of exercises and timing information within the video, such as through a user interface on a local application or an application available through the server (e.g., an app on a smart phone, a web page, cloud-based application, etc.).

At 1006, process 1000 can identify, for each exercise in the sequence of exercises, a group of potential corrections. In some embodiments, the group of potential corrections can include any suitable type of corrections. For example, in some embodiments, the group of potential corrections can include form corrections, such as positioning or relative positioning of anatomical points (e.g., feet not far enough apart, knees not bending with a large enough angle, and/or any other suitable type of positioning of anatomical points). As another example, in some embodiments, in an instance in which a particular exercise is to be repeated for a particular number of repetitions or for a particular duration of time, a potential correction in the group of potential corrections can include that the user is to speed up or maintain a particular pace.

In some embodiments, process 1000 can identify the group of potential corrections corresponding to a particular exercise in any suitable manner. For example, in some embodiments, process 1000 can query a database using the particular exercise as a key to retrieve a group of potential corrections corresponding to the particular exercise. In some such embodiments, a group of potential corrections for a particular exercise can be identified and stored in the database by any suitable user or entity. For example, in some embodiments, a group of corrections may be identified by an exercise identifier, a correction identifier, correction detection information (e.g., a set of anatomical criteria and thresholds), correction instructions, and other information. For example, a squat exercise may trigger a correction if a measured angle of a knee bend fails to exceed a threshold angle, and the correction instructions may include notifying the user was not sitting low enough.

In some embodiments, process 1000 provides a platform allowing instructors to upload exercise videos and provide pre-recorded feedback for common mistakes made by users when performing exercises. It is recognized that, for many exercises, users tend to make the same common mistakes allowing the instructor to identify, for example, three to five common corrections for each exercise to address most required feedback. In operation, the instructor uploads exercise content and pre-recorded video and/or audio for the most common error cases.

At 1008, process 1000 can cause a user interface to be presented on the instructor user device for verification of the exercises in the sequence of exercises and the group of potential corrections identified for each exercise. In various embodiments, the instructor can modify and verify the annotated data according to instructor preferences.

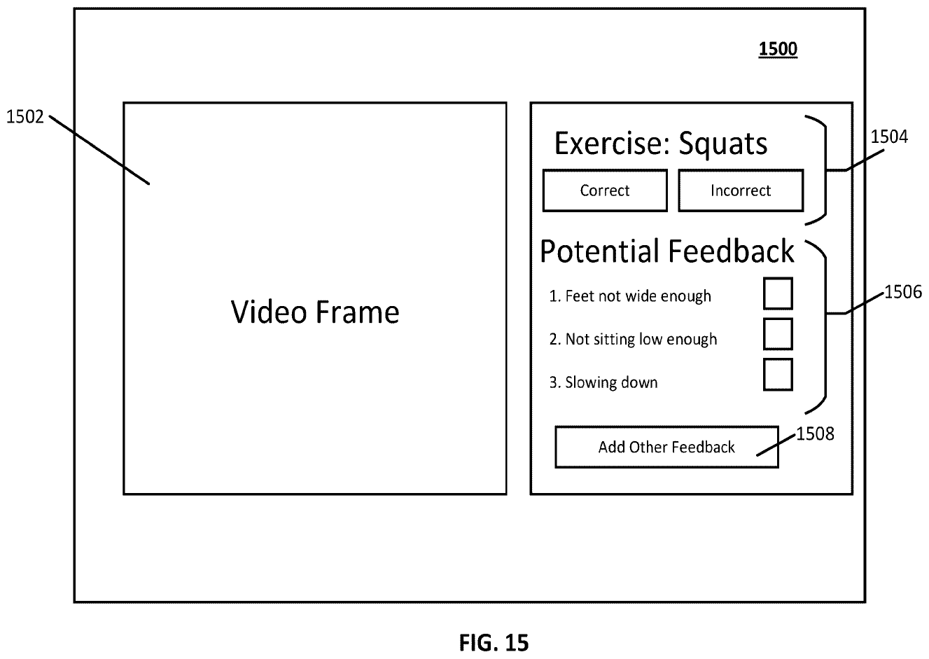

Turning to FIG. 15, an illustrative example 1500 of a user interface that can be provided to allow an instructor to verify an identified exercise and identified potential corrections is shown in accordance with some embodiments of the disclosed subject matter. As illustrated, in some embodiments, user interface 1500 can include a video frame 1502, an exercise confirmation portion 1504, a feedback confirmation portion 1506, and/or an add other feedback button 1508 (e.g. defining a new correction).

In some embodiments, video frame 1502 can include a frame or a series of frames of the video corresponding to the identified exercise. Note that, in instances in which video frame 1502 includes a series of frames of the video, the series of frames of the video can loop continually. Alternatively, in some embodiments, video frame 1502 can present a first frame of the series of frames, and the series of frames can be played in response to an input from a user of user interface 1500 (e.g., selection of a play button, and/or any other suitable input).

In some embodiments, exercise confirmation portion 1504 can include an indication of the exercise identified by process 1000 at 1004. In some embodiments, exercise confirmation portion 1504 can include one or more user interface controls (e.g., one or more push buttons, and/or any other suitable user interface controls) that can allow a user of user interface 1500 to confirm that the exercise identified by process 1000 is correct, modify the identified exercise, and/or delete the identified exercise.

In some embodiments, feedback confirmation portion 1506 can include the corrections in the group of potential corrections identified by process 1000 at 1006. In some embodiments, feedback confirmation portion 1506 can additionally include any suitable user interface controls that can allow a user of user interface 1500 to confirm that the identified potential corrections correspond to the identified exercise. For example, as shown in FIG. 15, each potential mistake can be associated with a check box that can allow a user of user interface 1500 to indicate that the potential mistake applies to the identified exercise. In some embodiments, the group of potential corrections in the feedback confirmation portion 1506 include a summary of the corrections and criteria for triggering the correction, including relative anatomical point locations and measured angles that can trigger the correction (e.g., feet too wide, not sitting low enough as measured by knee angles or position of hips relative to the knees), timing information and sequence of individual movements within the exercise, etc.

In some embodiments, add other feedback button 1508 can be any suitable user interface control (e.g., a push button, and/or any other suitable user interface control) that, in response to being selected, can cause another user interface to be presented for submitting other potential corrections and/or any other suitable additional information in connection with video frame 1502. Additionally or alternatively, in some embodiments, in response to selecting add other feedback button 1508, user interface 1500 can allow a user of user interface 1500 to record feedback that is presented to a user of device 100 upon identifying a feedback opportunity when the particular exercise is being performed (e.g., record an audio clip, record a video clip, input text feedback that is converted to audio data, etc.). For example, in response to selecting the potential feedback option of “Not sitting low enough” and selecting add other feedback button 1508, user interface 1500 can allow a user of user interface 1500 to record an audio clip in which the user states “For an even better squat, trying sitting lower until your thighs are parallel to the floor.” Upon identifying this correction when a user is performing the exercise in a video uploaded by an instructor, the corresponding audio clip can then be presented to the user.

Referring back to FIG. 10, at 1010, process 1000 can receive, from the instructor device, verification of and/or edits to the exercises in the sequence of exercises and/or the group of potential corrections. In some embodiments, process 1000 can receive the verification in the sequence of exercises and/or the group of potential corrections in any suitable manner. For example, in some embodiments, process 1000 can receive verification of a particular exercise and the group of potential corrections associated with the particular exercise via a user interface, such as the user interface shown in and described above in connection with FIG. 15. Note that, in some embodiments, process 1000 can iteratively cause a user interface to be presented for each exercise identified at 1004 and can receive verification for each exercise.

At 1012, process 1000 can store each verified exercise in connection with the timing information identified at 1004 and the verified group of potential corrections. In some embodiments, process 1000 can store the verified exercises, the verified group of potential corrections, and the timing information in any suitable manner. For example, in some embodiments, the verified exercises, the verified group of potential corrections, and the timing information can be stored in a file in connection with an identifier of the video.

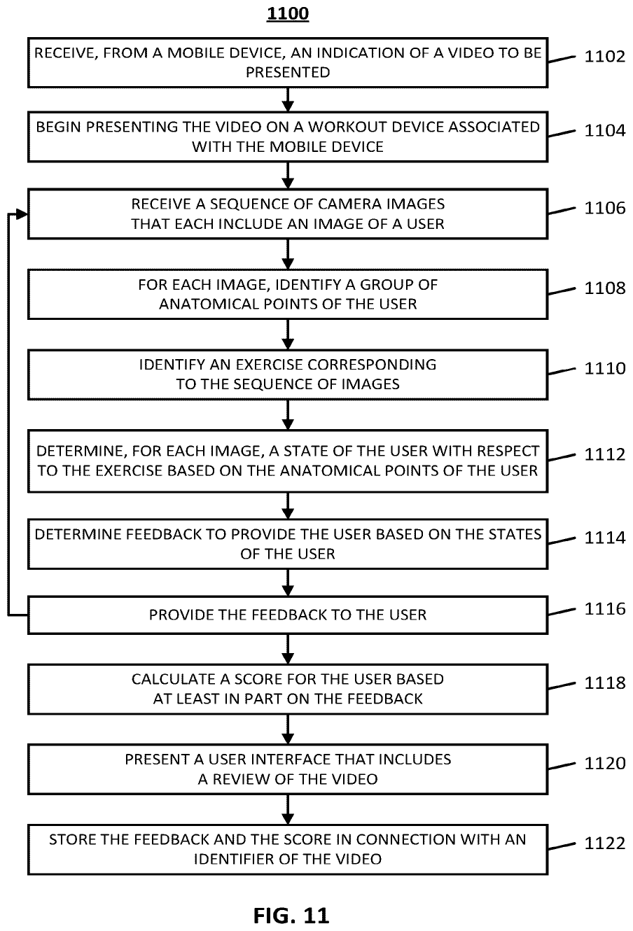

FIG. 11 is an illustrative example 1100 of a process for presenting an exercise video and feedback for a user in accordance with some embodiments of the disclosed subject matter. In some embodiments, process 1100 can be performed by any suitable device(s), such as a user device, a mobile device (e.g., a mobile phone, a tablet computer, a wearable computer, a laptop computer, and/or any other suitable mobile device) and/or a workout device (e.g., as shown in and described above in connection with FIGS. 1A, 1B, 2, 3, 4, 5, 6, 7, 8, and/or 9). Note that, in some embodiments, the mobile device can be paired with the workout device via any suitable communications link(s), such as BLUETOOTH, Wi-Fi, and/or any other suitable type of communications link(s).

Process 1100 can begin at 1102 by receiving, from the mobile device, an indication of a video to be presented. In some embodiments, the indication can be received by the mobile device in any suitable manner. For example, in some embodiments, the mobile device can present a user interface that allow a user of the mobile device to select a particular exercise video to be presented on a workout device paired with the mobile device.

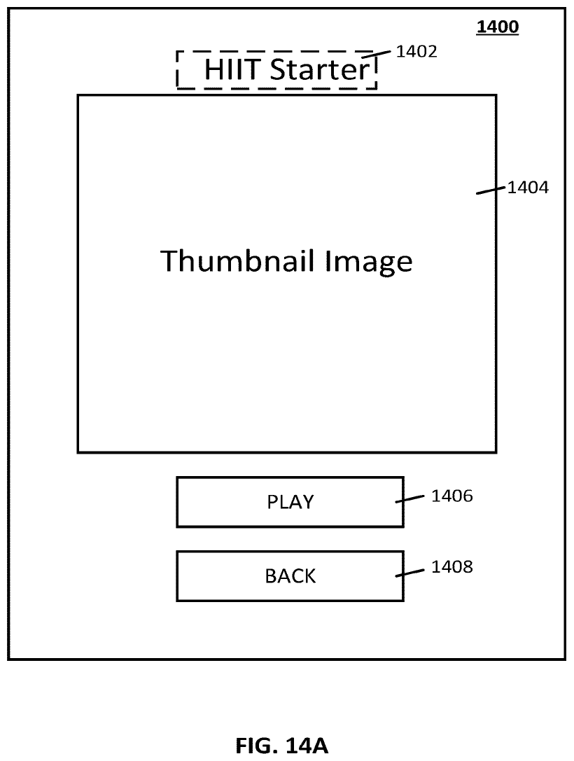

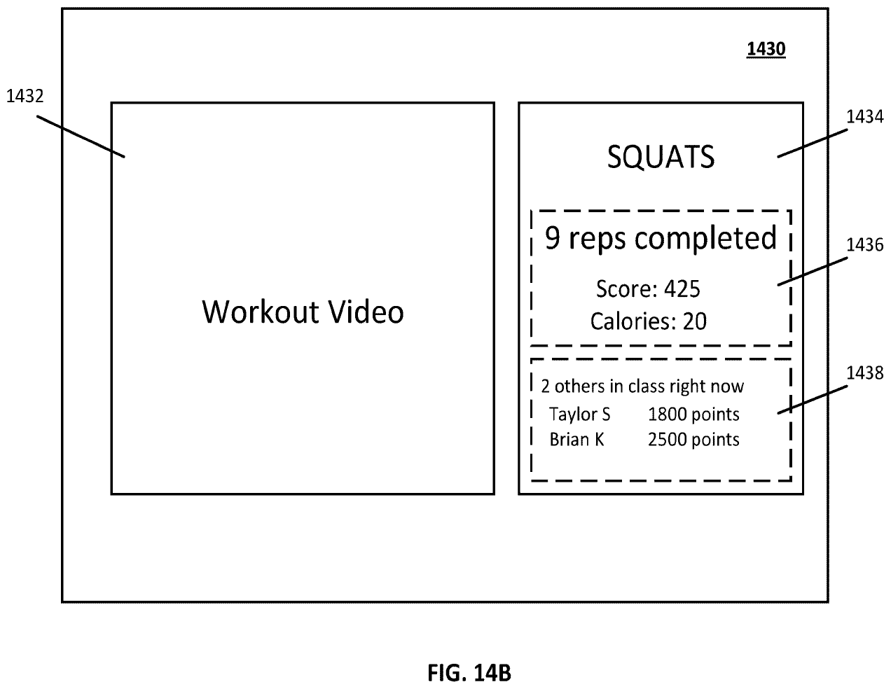

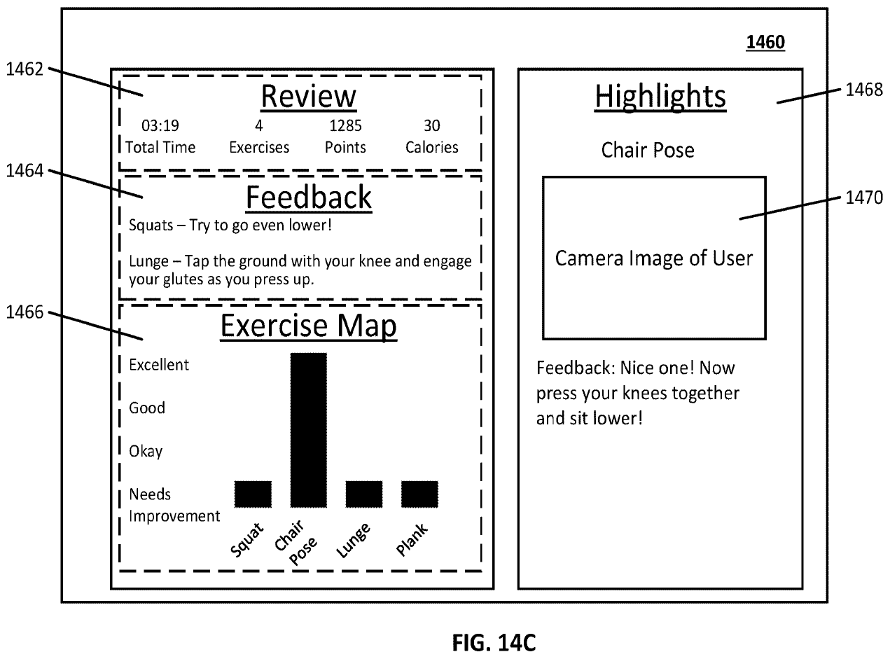

Turning to FIG. 14A, an illustrative example 1400 of a user interface for selecting a particular video is shown in accordance with some embodiments of the disclosed subject matter. As illustrated, user interface 1400 can include a video title 1402, a thumbnail image 1404, a play button 1406, and/or a back button 1408.

In some embodiments, video title 1402 can be any suitable title for a particular video. Note that, in some embodiments, user interface 1400 can include any other suitable text associated with the video, such as a name of an instructor of the video, a difficulty level of the video, a duration of the video, and/or any other suitable text.

In some embodiments, thumbnail image 1404 can be any suitable image associated with the video, such as a capture of a frame of the video, an image of an instructor of the video, and/or any other suitable image or icon.

In some embodiments, play button 1406 can be any suitable user interface control that, when selected, causes the video to begin being presented on a workout device that has been paired or is otherwise in communication with the mobile device.

In some embodiments, back button 1408 can be any suitable user interface control that, when selected, causes a previously presented user interface to be presented. For example, in some embodiments, selection of back button 1408 can cause a user interface that indicates available video items to be presented. As a more particular example, in some embodiments, the user interface can indicate available video content items that are recommended for a user of the user device based on videos previously viewed by the user. As another more particular example, in some embodiments, the user interface can indicate newly uploaded video content items.

Note that, in some embodiments, the indication of the video to be presented can be received directly by the workout device rather than being received by the mobile device, as described above. For example, in some embodiments, the workout device can present a user interface that indicates available video content items, and a user input can be received by the workout device (e.g., via a touchscreen display of the workout device, and/or in any other suitable manner). As another example, in some embodiments, the workout device can receive a spoken command (e.g., “play beginner yoga flow” and/or any other suitable command) via a microphone of the workout device.

Referring back to FIG. 11, at 1104, process 1100 can begin presenting the video on the workout device. In some embodiments, process 1100 can begin presenting the video on the workout device in any suitable manner. For example, in some embodiments, the workout device can stream the selected video from a server that hosts the video (e.g., the server described above in connection with FIG. 10, and/or any other suitable server). As a more particular example, in some embodiments, the mobile device can transmit an identifier of the selected video to the workout device, and the workout device can then request the selected video based on the identifier of the selected video from the server.

In some embodiments, process 1100 can present the video on a display of the workout device. For example, in some embodiments, the video can be presented on display 102 of device 100, as shown in and described above in connection with FIG. 1A. Note that, in some embodiments, the mobile device can be used to control playback of the video on the workout device. For example, in some embodiments, a user input to change a playback position of the video (e.g., rewind, fast-forward, and/or any other suitable change in playback position) can be received by the mobile device and can be transmitted to the workout device. As another example, a user input to change a volume of audio content associated with the video can be received by the mobile device and can be transmitted to the workout device.

Note that, in some embodiments, audio content associated with the video can be presented in any suitable manner. For example, in some embodiments, audio content can be presented via a speaker of the workout device. In some such embodiments, the speaker can be located at any suitable position of the workout device, such as on a back panel of the workout device, on a front panel of the workout device, inside the workout device, and/or at any other suitable position.

At 1106, process 1100 can receive a sequence of camera images that each include an image of a user of the workout device. In some embodiments, each image in the sequence of camera images can be captured by a camera associated with the camera device, such as by camera 210 on camera arm 106, as shown in and described above in connection with FIG. 2. Note that, in some embodiments, the workout device can be placed on or attached to a top portion of a mat (e.g., a yoga mat, and/or any other suitable type of exercise mat), and the camera can be configured at an angle that allows an image captured by the camera to include the mat, thereby allowing each image to include a user on the mat. In some embodiments, any suitable number of camera images can be captured (e.g., one, five, ten, twenty, and/or any other suitable number). In some embodiments, there can be any suitable time duration between successively captured camera images (e.g., half a second, one second, two seconds, and/or any other suitable duration).

At 1108, process 1100 can identify a group of anatomical points of the user for each image. In some embodiments, the anatomical points of the user can include any suitable points, such as ankles, feet, knees, shoulders, hips, a top of the head, hands, and/or any other suitable points. In some embodiments, process 1100 can identify the group of anatomical points of the user using any suitable technique(s). For example, in some embodiments, process 1100 can use any suitable image recognition technique(s) to identify locations of each of the anatomical points of the user. Note that, in some embodiments, any suitable number of anatomical points can be identified (e.g., two, five, ten, fifteen, twenty, and/or any other suitable number). In some embodiments, the anatomical points are determined using an image classification system, which may include a trained neural network.

At 1110, process 1100 can identify an exercise corresponding to the sequence of images. In some embodiments, process 1100 can identify the exercise corresponding to the sequence of images using any suitable technique(s). For example, in some embodiments, process 1100 can identify timing information corresponding to the video that indicates an exercise corresponding to a time range of the sequence of images. In some embodiments, the timing information can correspond to the timing information generated by process 1000 as shown in and described above in connection with FIG. 10.

At 1112, process 1100 can determine, for each image in the sequence of images, a state of the user with respect to the identified exercise based on the anatomical points. In some embodiments, the state of the user can correspond to a dynamic position of the user during performance of the identified exercise. Note that, in some embodiments, an exercise can be identified with a group of states that the user can be in during performance of the exercise, and, in some embodiments, process 1100 can identify, for a particular exercise, a state the user is in for a particular image of the sequence of images from the group of states associated with the exercise. For example, in an instance in which the identified exercise corresponding to the sequence of images is a squat, the group of states can include: 1) a standing position; 2) an initial bent-knee position; 3) a lowest point position; and 4) a bent-knee position as the user returns to standing. Continuing with this example, in some embodiments, process 1100 can identify, for each image, a state of the user based on the anatomical points of the user in the image. Continuing further with this example, in an instance in which a first image of the sequence of images has anatomical points that correspond to a user standing fully upright, process 1100 can determine that the state of the user with respect to the squat exercise is State 1. Continuing still further with this example, in an instance in which the second image of the sequence of images has anatomical points that correspond to the user with slightly bent knees, process 1100 can determine that the state of the user with respect to the squat exercise is State 2. In some embodiments, process 1100 can iterate through each of the images in the sequence of images and can assign a state corresponding to the identified exercise to each image based on anatomical points of the user identified in each image.

In some embodiments, the state of the user is determined by analysis of the image through a trained neural network classifier. In some embodiments, the relative positions of the anatomical points are analyzed to determine a current state. For example, during a squat the user moves from a standing position, in which the legs and hips are substantially straight to a lowest point, where the person is in a sitting position. Thus, the states in a squat can be determined based on the anatomical points by estimating the current angle of the knees and hips during the movement. In some embodiments, if a next state in an exercise sequence is not achieved by the user, the system can send a correction to the user with instructions on proper form and execution of the exercise.

Note that, in some embodiments, different exercises can have different states. Additionally, note that, in some embodiments, different exercises can have a different number of states. In some embodiments, any suitable number of states (e.g., one, two, five, ten, and/or any other suitable number) can be associated with a particular exercise.

At 1114, process 1100 can determine feedback to provide the user based on the states of the user with respect to the identified exercise. In some embodiments, the feedback can include any suitable feedback. For example, in some embodiments, the feedback can include a suggested correction with respect to one or more states of the user. As a more particular example, in an instance in which the identified exercise is a squat, process 1100 can determine that for a state corresponding to a lowest point of the squat (e.g., State 3 as described in the example described above in connection with block 1112), the suggested correction is that the user should try to sit lower. In this example, the anatomical points of State 3 may be analyzed to determine the depth achieved at the lowest point in the squat, for example by determining the angle of the femur with respect to the floor and triggering a correction if the angle fails to go below a predetermined threshold angle for the state.

As another more particular example, in an instance in which the identified exercise is a lunge, process 1100 can determine that for a state corresponding to an initial starting position, the suggested correction is that the user should take a wider stance with their feet. As another example, in some embodiments, the feedback can include a suggested correction relating to a pace at which the user is performing the exercise. As a more particular example, in an instance in which the identified exercise is an exercise that is respectively performed (e.g., jumping jacks, a series of squats, and/or any other suitable repetitively performed exercise), the suggested correction can be that the user should maintain a particular pace. In various embodiments, the suggested correction(s) may be triggered by tracking one or more variables (e.g., exercise states as determined by measurements estimated from the anatomical points, the pace of repetitions, etc.) and comparing the tracked value to a minimum performance threshold.

In some embodiments, process 1100 can determine the feedback to provide the user in any suitable manner. For example, in some embodiments, for a particular image of the sequence of images, process 1100 can identify a relative positioning of anatomical points of the user within the image with respect to other anatomical points and can determine the feedback based on the relative positioning and the identified state corresponding to the exercise. As a more particular example, in an instance in which the exercise is a lunge and the state corresponding to the image is an initial starting point, process 1100 can identify a distance between two anatomical points corresponding to each of the feet of the user. Continuing with this particular example, process 1100 can compare the distance of the feet of the user to any suitable ground truth distance for a correct lunge positioning, and can determine whether the distance of the feet of the user in the image is within a predetermined range (e.g., within six inches, within twelve inches, and/or any other suitable predetermined range) of the ground truth distance. Continuing further with this particular example, in an instance in which process 1100 determines that the distance of the feet of the user is not within the predetermined range of the ground truth distance, process 1100 can determine that the feedback is to be to change the distance of the feet of the user to be closer to the ground truth distance (e.g., “widen your stance,” “narrow your stance,” and/or any other suitable feedback). As another more particular example, in some embodiments, process 1100 can determine the feedback based on whether a particular anatomical point is higher, lower, or in a line with any other suitable anatomical points. As a specific example, in an instance in which the exercise is a plank, process 1100 can determine whether anatomical points corresponding to hips of the user or the lower back of the user is above, below, or in a line with any other suitable anatomical points, such as the shoulders of the user. Continuing with this specific example, in some embodiments, process 1100 can identify feedback that causes the anatomical points to be brought into correct alignment based on a ground truth alignment (e.g., “bring your hips lower,” and/or any other suitable feedback).

As another example, in some embodiments, process 1100 can identify the feedback by determining an angle between one or more joints based on anatomical points of the user in a particular image, and by comparing the angle of the one or more joints to a ground truth angle to determine whether the angle of the one or more joints are within a predetermined range to the ground truth angle (e.g., within five degrees, within ten degrees, and/or any other suitable range). As a more particular example, in an instance in which the exercise is a squat and in which the state of the user corresponding to the image is a state in which the user is fully in the squat position, process 1100 can determine an angle of a knee joint of the user and/or an angle of the hip joint of the user based on any suitable anatomical points (e.g., a position of an ankle of the user, a position of a knee of the user, a position of a hip of the user, and/or any other suitable anatomical points). Continuing with this particular example, in an instance in which process 1100 determines that an angle of a knee joint of the user is 60 degrees, and that a ground truth angle of the knee joint corresponding to the particular state is 90 degrees, process 1100 can determine that the angle of the knee joint of the user is not within the predetermined range of the ground truth angle of the knee joint for the particular state of the exercise, and can therefore determine that the feedback is to increase the angle of the knee joint (e.g., “bend your knees more,” and/or any other suitable feedback).

As yet another example, in some embodiments, process 1100 can identify the feedback based on a positioning of one or more anatomical points with respect to the mat the user is performing the exercise on. As a more particular example, in an instance in which the exercise is a lunge and in which the state corresponding to the image is a lowest point of the lunge, process 1100 can determine whether a knee of the user is touching the mat and/or is relatively close to the mat. Continuing further with this example, in an instance in which process 1100 determines that the knee of the user is not touching the mat and/or is not within a predetermined distance to the mat, process 1100 can identify feedback that causes the user to touch the mat (e.g., “tap the mat with your knee,” and/or any other suitable feedback).

In some embodiments, in instances in which process 1100 identifies the feedback based on ground truth information, process 1100 can identify the ground truth information in any suitable manner. For example, in some embodiments, process 1100 can retrieve ground truth information from a database that stores potential feedback for different states of different exercises provided by an instructor or other entity, such as described above in connection with 1006 of FIG. 10.

Note that, in some embodiments, process 1100 can identify multiple feedback items for a particular exercise and/or for a particular state of an exercise.

At 1116, process 1100 provide the feedback to the user. In some embodiments, process 1100 can provide the feedback in any suitable manner. For example, in some embodiments, process 1100 can provide the feedback as spoken audio output provided via a speaker used to present audio content associated with the video. In some such embodiments, process 1100 can lower a volume of other audio content associated with the video (e.g., background music, other instructions provided by the instructor of the video, and/or any other suitable audio content) to provide the feedback. Note that, in some embodiments, spoken feedback can be provided in a voice of the instructor of the video using any suitable technique or combination of techniques. For example, in some embodiments, spoken feedback relating to common corrections for a particular exercise can be pre-recorded by the instructor, and process 1100 can retrieve the pre-recorded spoken feedback corresponding to the feedback identified at 1114 for presentation to the user. As another example, in some embodiments, process 1100 can provide the feedback as a visual user interface presented on the display of the workout device. As a more particular example, in some embodiments, process 1100 can present the feedback as a pop-up overlay superimposed on the video content. In some such embodiments, process 1100 can cause the user interface to be inhibited from presentation after any suitable duration of time has elapsed (e.g., five seconds, ten seconds, and/or any other suitable duration of time).

In some embodiments, process 1100 can determine whether feedback is to be presented to the user during presentation of the video and/or a manner in which the feedback is to be presented to the user based on user-configured settings. For example, in some embodiments, the user-configured settings can indicate that full feedback is to be given, as spoken feedback, as a visual overlay, and/or in any other suitable manner. As another example, in some embodiments, the user-configured settings can indicate that an audible tone is to be presented in response to determining that feedback has been identified, and that the feedback is to be presented in response to a request for the feedback from the user. As yet another example, in some embodiments, the user-configured settings can indicate that feedback is to be saved until the user has completed the workout and is to be presented after the video has finished (e.g., within a user interface presented in response to determining that the video has finished, and/or in any other suitable manner). In some embodiments, a manner in which feedback is presented can be adjusted automatically over time. For example, in some embodiments, all feedback can be initially presented, and, after a user has completed more than a predetermined number of exercise videos (e.g., more than ten, more than twenty, and/or any other suitable number), feedback can be collected and presented at the end of a video.

Turning to FIG. 14B, an illustrative example 1430 of a user interface that includes presentation of the video and feedback for the user is shown in accordance with some embodiments of the disclosed subject matter. As illustrated, in some embodiments, user interface 1430 can include video content 1432, and a feedback panel 1434.

In some embodiments, video content 1432 can be a presentation of the exercise video.

In some embodiments, feedback panel 1434 can include an exercise feedback section 1436. In some embodiments, exercise feedback section 1436 can include any suitable information, such as a number of repetitions of a current exercise that have been completed, an indication of the feedback for the exercise as described above in connection with block 1112 (e.g., “try and get lower,” “bend your knees more,” “keep up the pace,” and/or any other suitable feedback), a score for the current exercise and/or a total current score for the video (e.g., as described below in connection with block 1118), a total number of calories burned at a current time of the video, and/or any other suitable information.

In some embodiments, feedback panel 1434 can additionally include an “other participants” section 1438, as shown in FIG. 14B. In some embodiments, other participants section 1438 can include identifiers of other users currently viewing and/or participating in the same video (e.g., names of the other users, icons representing the other users, and/or any other suitable user information). Additionally, in some embodiments, other participants section 1438 can include current scores for each user indicated in other participants section 1438. Note that, additional details related to techniques to calculate a score for a video are described below in more detail in connection with block 1118. Additionally, note that, in some embodiments, other participants section 1438 can be presented in response to determining that other users are currently viewing or participating in the same video. For example, in some embodiments, other participants section 1438 can be presented in instances in which the video is being presented is a live-streamed video. In some embodiments, the other participants section may display archived data from users who previously participated in the exercise session to simulate a live exercise class experience for the user. Additionally, or alternatively, in some embodiments, other participants panel 1438 can be omitted.

In some embodiments, process 1100 can loop back to 1106 and can receive a new sequence of images. In some embodiments, process 1100 can loop through blocks 1106-1116 during presentation of the video to identify feedback for each exercise included in the video until presentation of the video has finished.

At 1118, process 1100 can calculate a score for the user for the video based at least in part on the feedback. In some embodiments, the score can indicate how well the user performed the exercises included in the video. In some embodiments, process 1100 can calculate the score in any suitable manner. For example, in some embodiments, process 1100 can calculate a score for the user for each exercise included in the video, and can calculate the score for the user for the video by aggregating the score for each exercise in any suitable manner (e.g., by summing the scores for each exercise, by calculating an average or a weighted average of the scores for each exercise, and/or in any other suitable manner). As another example, in some embodiments, process 1100 can calculate the score for the video based on an amount of the video the user completed. As a more particular example, in some embodiments, process 1100 can calculate the score such that the score is relatively higher in instances in which the user completes more of the video.

In some embodiments, process 1100 can calculate the score for each exercise based on any suitable information. For example, in some embodiments, process 1100 can calculate the score based on a number of repetitions completed for the exercise such that the score is directly correlated with a number of completed repetitions. As another example, in some embodiments, process 1100 can calculate the score for the exercise based on the suggested corrections provided in the feedback for the exercise and/or based on a number of suggested corrections. As a more particular example, in some embodiments, process 1100 can calculate the score for the exercise such that the user receives a maximum possible score for the exercise if no suggested corrections were identified. Continuing with this particular example, in some embodiments, process 1100 can calculate the score for the exercise such that the score is less than the maximum possible score if a suggested correction was identified for the exercise.

At 1120, process 1100 can present a user interface that includes a review of the video.

Turning to FIG. 14C, an illustrative example 1460 of a user interface that includes a review of the video is shown in accordance with some embodiments of the disclosed subject matter. As illustrated, in some embodiments, user interface 1460 can include a review panel 1462, a feedback panel 1464, an exercise map 1466, and/or a highlights panel 1468.

In some embodiments, review panel 1462 can include any suitable information about the video and/or a performance of the user during presentation of the video. For example, as shown in FIG. 14C, in some embodiments, review panel 1462 can include a duration of the video and/or a duration of the video completed by the user, a number of exercises included in the video, a score for the user during completion of the video (e.g., the score calculated as described above in connection with 1118), and/or a number of calories estimated to be burned by the user during completion of the exercises included in the video.

In some embodiments, feedback panel 1464 can include any suitable feedback for any exercises included in the video. For example, in some embodiments, feedback panel 1464 can include any of the feedback identified at block 1114, as described above.

In some embodiments, exercise map 1466 can indicate scores for each of the exercises included in the video. For example, as shown in FIG. 14C, exercise map 1466 can include a bar graph or other visual representation of scores for each exercise included in the video. Note that, although exercise map 1466 shows scores for each exercise mapped to four categories (“excellent,” “good,” “okay,” and “needs improvement”), in some embodiments, exercise map 1466 can indicate a numerical score for each exercise, for example, the score calculated for each exercise as described above in connection with 1118 of FIG. 11.

In some embodiments, highlights panel 1468 can show any suitable information associated with an exercise identified to have been performed particularly well by the user. For example, in some embodiments, highlights panel 1468 can include a camera image 1470 that includes an image of the user performing the exercise. In some embodiments, camera image 1470 can be any suitable image. For example, in some embodiments, camera image 1470 can be an image included in the sequence of images described above in connection 1106 of FIG. 11.

Note that, in some embodiments, the exercise associated with highlights panel 1468 can be identified in any suitable manner. For example, in some embodiments, the exercise can be identified as the exercise associated with the highest score. In some embodiments, camera image 1470 can be identified in any suitable manner. For example, in some embodiments, camera image 1470 can be identified as an image for which no suggested corrections and/or relatively few suggested corrections were identified, as described above in connection with 1114 of FIG. 11.

Referring back to FIG. 11, in some embodiments, the user interface that includes the review of the video can be presented on any suitable device. For example, in some embodiments, process 1100 can cause the user interface to be presented on a mobile device of the user (e.g., the mobile device from which the video was selected, as described above in connection with 1102). As another example, in some embodiments, process 1100 can cause the user interface to be presented on the workout device.

At 1122, process 1100 can store the feedback identified for exercises included in the video, the scores calculated for exercises included in the video, and the score for the video in connection with an identifier of the video. In some embodiments, the information can be stored in connection with a user account associated with the user such that the user can access the stored information via the user account from any suitable user device. In some embodiments, the information can be stored locally on the workout device and/or on the mobile device paired with the workout device. Additionally, or alternatively, in some embodiments, process 1100 can transmit the information to a server (e.g., a server associated with an exercise video service from which the video is provided, and/or any other suitable server) for storage on the server in connection with the user account.

Note that, in some embodiments, a video can be live-streamed by a user. In some such embodiments, feedback identified by process 1100 can be transmitted to an instructor device associated with an instructor of the exercise video. In some embodiments, the feedback can be used to allow the instructor to generate a comment (e.g., a spoken audio comment, and/or any other suitable comment) that is to be presented by the workout device. For example, in some embodiments, process 1100 can transmit feedback that indicates that process 1100 has determined that a user is not bending the knees enough. Continuing with this example, in some embodiments, an instructor can receive the feedback through an instructor device used to live-stream the exercise video, and can record a comment (e.g., “be sure to bend lower,” etc.) that is to be presented to the user by the workout device. In some such embodiments, process 1100 can transmit the identified feedback to the instructor device in connection with an identifier of the user (e.g., in connection with a name of the user, and/or any other suitable identifier), thereby allowing the instructor to generate a comment personalized to the user.

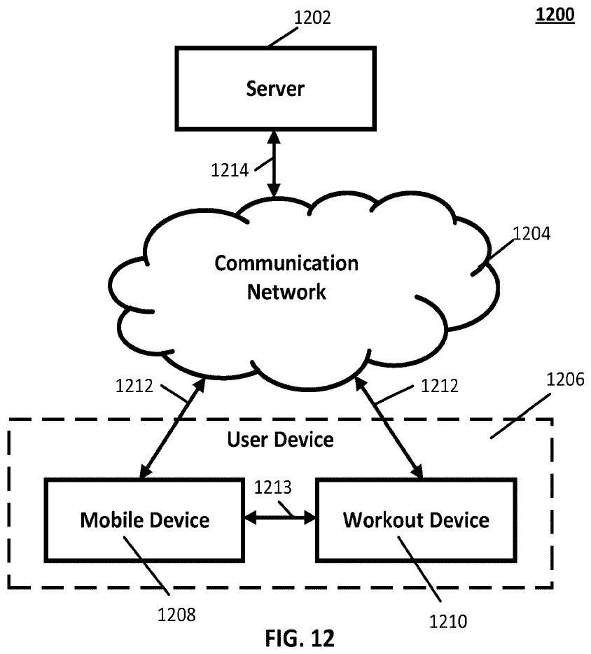

Turning to FIG. 12, an illustrative example 1200 of hardware for presenting exercise videos with feedback that can be used in accordance with some embodiments of the disclosed subject matter is shown. As illustrated, hardware 1200 can include a server 1202, a communication network 1204, and/or one or more user devices 1206, such as mobile device 1208 and workout device 1210.

Server 1202 can be any suitable server(s) for storing information, data, programs, videos, user account information, and/or any other suitable content. In some embodiments, server 1202 can perform any suitable function(s). For example, in some embodiments, server 1202 can cause an exercise video to begin being presented on a display of workout device 1210. As another example, in some embodiments, server 1202 can store information associated with a user account, such as exercise videos a user corresponding to the user account has previously completed, scores for a user of the user account for previously completed exercise videos, feedback previously given to a user of the user account, and/or any other suitable user account information. As yet another example, in some embodiments, server 1202 can store any suitable programs used to identify feedback to be provided to a user of workout device 1210 based on camera information captured from a camera of workout device 1210. In some such embodiments, server 1202 can transmit such a program or algorithm to workout device 1210.

Communication network 1204 can be any suitable combination of one or more wired and/or wireless networks in some embodiments. For example, communication network 1204 can include any one or more of the Internet, an intranet, a wide-area network (WAN), a local-area network (LAN), a wireless network, a digital subscriber line (DSL) network, a frame relay network, an asynchronous transfer mode (ATM) network, a virtual private network (VPN), and/or any other suitable communication network. User devices 1206 can be connected by one or more communications links (e.g., communications links 1212) to communication network 1204 that can be linked via one or more communications links (e.g., communications links 1214) to server 1202. The communications links can be any communications links suitable for communicating data among user devices 1206 and server 1202 such as network links, dial-up links, wireless links, hard-wired links, any other suitable communications links, or any suitable combination of such links.

In some embodiments, mobile device 1208 can communicate with workout device 1210 for any suitable purpose(s). For example, in some embodiments, mobile device 1208 can be used to select a particular exercise video, which can then be presented on a display of workout device 1210. In some embodiments, mobile device 1208 can be any suitable type of mobile device, such as a mobile phone, a tablet computer, a wearable computer, a laptop computer, and/or any other suitable type of mobile device.

In some embodiments, mobile device 1208 can communicate with workout device 1210 via one or more communications links (e.g., communications link 1213). In some embodiments, communications link 1213 can be any suitable type of communications link, such as BLUETOOTH, Wi-Fi, and/or any other suitable type of communications link.

In some embodiments, workout device 1210 can be a device such as device 100 shown in and described above in connection with FIGS. 1A, 1B, 2, 3, 4, 5, 6, 7, 8, and/or 9.

Although server 1202 is illustrated as one device, the functions performed by server 1202 can be performed using any suitable number of devices in some embodiments. For example, in some embodiments, multiple devices can be used to implement the functions performed by server 1202.

Although one mobile device 1208 and one workout device 1210 are shown in FIG. 12 to avoid over-complicating the figure, any suitable number of user devices or workout devices, and/or any suitable types of user devices, can be used in some embodiments. For example, mobile device 1208 and workout device 1210 may be combined into a single device performing the functions of the mobile device 1208 and workout device 1210 as described herein.

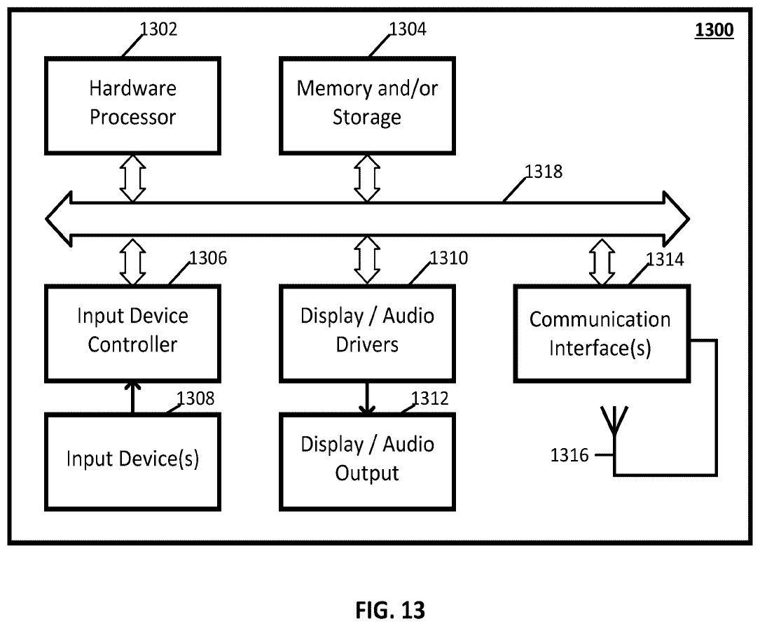

Server 1202 and user devices 1206 can be implemented using any suitable hardware in some embodiments. For example, in some embodiments, devices 1202 and 1206 can be implemented using any suitable general-purpose computer or special-purpose computer. For example, a mobile phone may be implemented using a special-purpose computer. Any such general-purpose computer or special-purpose computer can include any suitable hardware. For example, as illustrated in example hardware 1300 of FIG. 13, such hardware can include hardware processor 1302, memory and/or storage 1304, an input device controller 1306, an input device 1308, display/audio drivers 1310, display and audio output circuitry 1312, communication interface(s) 1314, an antenna 1316, and a bus 1318.

Hardware processor 1302 can include any suitable hardware processor, such as a microprocessor, a micro-controller, digital signal processor(s), dedicated logic, and/or any other suitable circuitry for controlling the functioning of a general-purpose computer or a special-purpose computer in some embodiments. In some embodiments, hardware processor 1302 can be controlled by a server program stored in memory and/or storage of a server, such as server 1202. In some embodiments, hardware processor 1302 can be controlled by a computer program stored in memory and/or storage 1304 of workout device 1210.

Memory and/or storage 1304 can be any suitable memory and/or storage for storing programs, data, and/or any other suitable information in some embodiments. For example, memory and/or storage 1304 can include random access memory, read-only memory, flash memory, hard disk storage, optical media, and/or any other suitable memory.

Input device controller 1306 can be any suitable circuitry for controlling and receiving input from one or more input devices 1308 in some embodiments. For example, input device controller 1306 can be circuitry for receiving input from a touchscreen, from a keyboard, from one or more buttons, from a voice recognition circuit, from a microphone, from a camera, from an optical sensor, from an accelerometer, from a temperature sensor, from a near field sensor, from a pressure sensor, from an encoder, a heart rate monitor, and/or any other type of input device. In some embodiments, the input device controller 1306 is configured to communicate with a smart watch configured to sense and track user fitness information for integration into the performance feedback system.

Display/audio drivers 1310 can be any suitable circuitry for controlling and driving output to one or more display/audio output devices 1312 in some embodiments. For example, display/audio drivers 1310 can be circuitry for driving a touchscreen, a flat-panel display, a cathode ray tube display, a projector, a speaker or speakers, and/or any other suitable display and/or presentation devices.

Communication interface(s) 1314 can be any suitable circuitry for interfacing with one or more communication networks (e.g., computer network 1204). For example, interface(s) 1314 can include network interface card circuitry, wireless communication circuitry, and/or any other suitable type of communication network circuitry.

Antenna 1316 can be any suitable one or more antennas for wirelessly communicating with a communication network (e.g., communication network 1204) in some embodiments. In some embodiments, antenna 1316 can be omitted.

Bus 1318 can be any suitable mechanism for communicating between two or more components 1302, 1304, 1306, 1310, and 1314 in some embodiments.

Any other suitable components can be included in hardware 1300 in accordance with some embodiments.

In some embodiments, at least some of the above described blocks of the processes of FIGS. 10 and 11 can be executed or performed in any order or sequence not limited to the order and sequence shown in and described in connection with the figures. Also, some of the above blocks of FIGS. 10 and 11 can be executed or performed substantially simultaneously where appropriate or in parallel to reduce latency and processing times. Additionally, or alternatively, some of the above described blocks of the processes of FIGS. 10 and 11 can be omitted.

In some embodiments, any suitable computer readable media can be used for storing instructions for performing the functions and/or processes herein. For example, in some embodiments, computer readable media can be transitory or non-transitory. For example, non-transitory computer readable media can include media such as non-transitory forms of magnetic media (such as hard disks, floppy disks, and/or any other suitable magnetic media), non-transitory forms of optical media (such as compact discs, digital video discs, Blu-ray discs, and/or any other suitable optical media), non-transitory forms of semiconductor media (such as flash memory, electrically programmable read-only memory (EPROM), electrically erasable programmable read-only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

FIGS. 31A and 31B show examples of a user 3002 exercising on a mat 208 in connection with a device for presenting exercise content and detecting user performance information, in accordance with some embodiments of the disclosed subject matter. In these examples, the mat 208 identifies an exercise space in front of the display and camera 206, where the user 3002 can see the workout device 100, including the user interface screen 1430, and have the user's exercise activity captured on the camera 206 for feedback monitoring and corrections. In some embodiments, the workout device 100 includes an initialization screen to ensure proper set up, orientation and alignment of the device 100, the mat 208 and the camera 210 before exercise. For example, the workout device 100 display a view from the camera and provide instructions for positioning the camera such that the mat 208 is visible in the camera images. The workout device 100, may further instruct the user on a proper exercise distance from the display and camera.

In some embodiments, the mat 208 includes one or more haptic response components (e.g., vibrating motors) communicably coupled to the workout device 100 and configured to provide feedback to the user 3002 from the workout device 100, which may include positive feedback (e.g., indicating the user 3002 is using proper form) or negative feedback (e.g., indicating the user 3002 needs to correct form). In some embodiments, the mat 208, includes sensors configured to sense pressure from the user identifying portions of the mat 208 being occupied by the user 3002. For example, the mat 208 may be communicably coupled to the workout device 100 and provide performance information such as number of steps and/or foot locations on the mat 208 to assist in accurately tracking the user's anatomical points.

Note that, although the present disclosure illustrates an example mat-based exercise system, the system and methods disclosed herein may be used to analyze user performance and provide corrective instructions in other exercise environments and with other exercise equipment, including but not limited to, weightlifting exercises (free weights and machines), treadmill operation, cycling, and/or rowing.

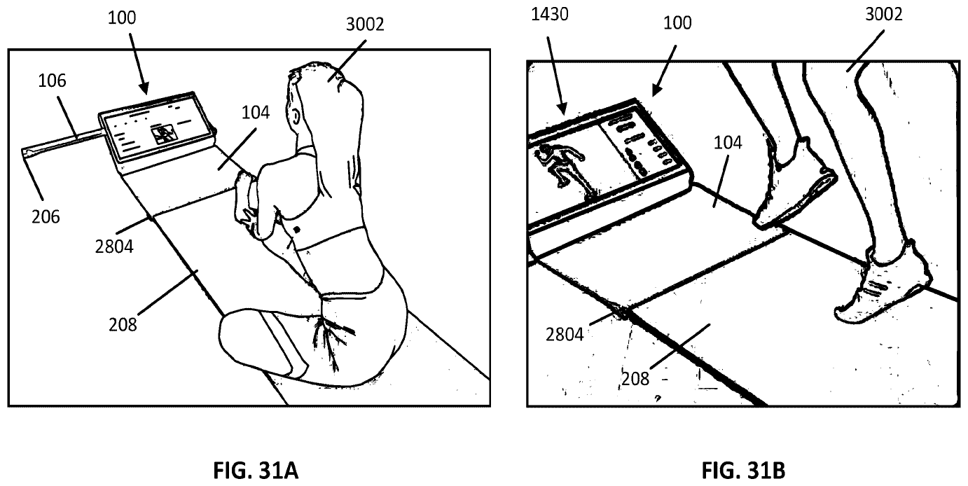

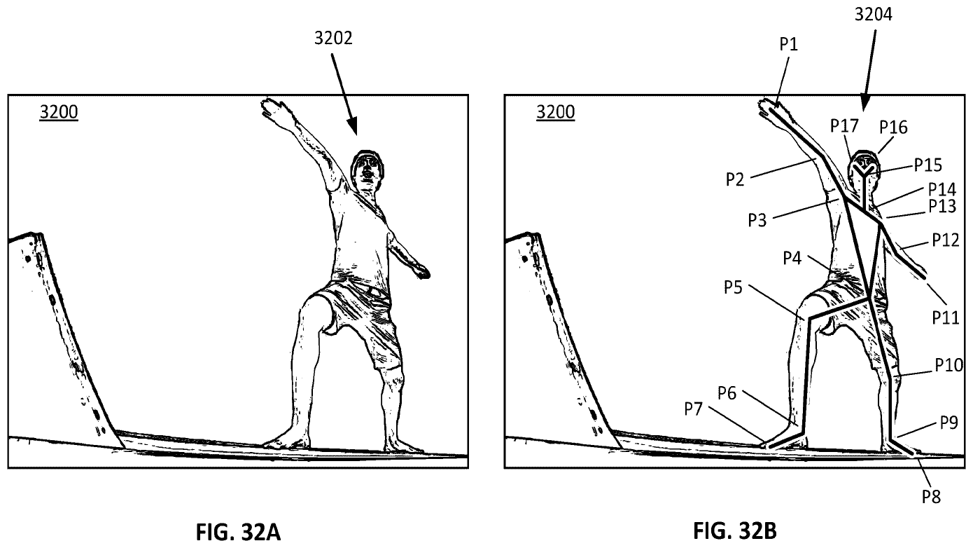

FIGS. 32A and 32B show an example of a camera image 3200 and associated anatomical points P1-P17, in accordance with some embodiments of the disclosed subject matter. In some embodiments, the camera captures one or more images 3200 of the user 3202 and facilitates anatomical point processing (e.g., processing the image 3200 on the workout device, sending the image 3200 to a server for processing, etc.). In various embodiments, a plurality of anatomical points may be identified on the user 3202 in the image 3200. In various embodiments, different numbers of points representing difference anatomical features may be used. In the illustrated embodiment, the anatomical points include: right hand (P1), right elbow (P2), right shoulder (P3), center hip (P4), right knee (P5), right ankle (P6), right foot (P7), left foot (P8), left ankle (P9), left knee (P10), left hand (P11), left elbow (P12), left shoulder (P13), center torso P14, nose (P15), left eye (P16) and right eye (P17). The anatomical points P1-P17 form a wireframe 3204 representation of the user's body position. The anatomical points P1-P17 can be used to determine the relative location of each anatomical point P1-P17, the position and orientation of each line segment between a pair of anatomical points, and angles between line segments. Analysis of anatomical point information allows the system to measure exercise performance, determine a current stage of an exercise, identify corrections, and perform other functions associated with the user's body position.

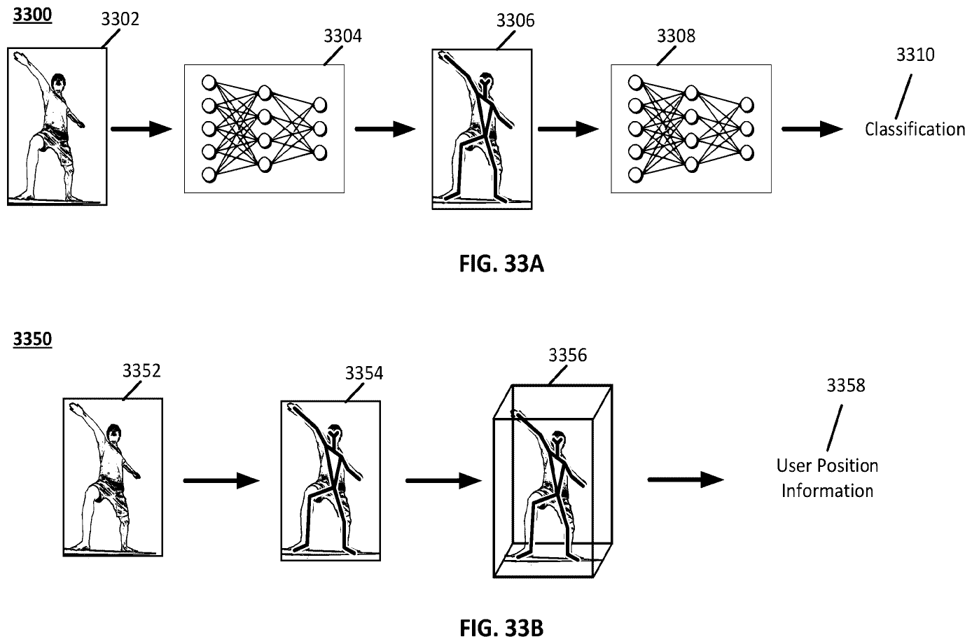

Referring to FIGS. 33A and 33B, example processes for image and anatomical point processing will now be described, in accordance with some embodiments of the disclosed subject matter. In various embodiments, any suitable technique for identifying anatomical points from an image and classifying the results may be used. In FIG. 33A, an example process 3300 includes receiving one or more images 3302 of a user during exercise for processing. In some embodiments, the user is positioned on a mat at a known distance and orientation relative to the workout device. The image 3302 is processed to identify anatomical positions (e.g., points P1-P17 of FIG. 32B). In some embodiments, the image of the user is input to one or more trained convolutional neural networks (CNN) 3304 to output the desired anatomical points 3306. In some embodiments, the CNNs are trained using labeled training images of various users during workouts. In various embodiments, the anatomical points 3306 may represent the location of the points on the two-dimensional image or in three-dimensional space (e.g., relative to the camera).

The anatomical points 3306 from one or more images (e.g., a sequence of images representing the user executing an exercise) are then processed to determine a stage of the exercise (e.g., as previously described herein) and/or determine whether correction is appropriate through another suitable method. In some embodiments, the anatomical point data 3306 is input to one or more trained CNNs 3308 to classify the stage of exercise, determine whether corrections are desired, and/or determine other user exercise information.

Another example of image and anatomical point processing is illustrated in FIG. 33B, which will now be described in accordance with some embodiment of the disclosed subject matter. In process 3305, an image 3352 received from camera of a workout device is received for processing and anatomical points are determined on the two-dimensional image 3354. The anatomical points may be determined through any suitable machine vision process, such as using as active appearance model algorithms, active shape model algorithms, trained neural networks, and/or other processing technique. The two-dimensional data 3354 is then mapped to a three-dimensional space 3356 through one or more two-dimension to three-dimension conversion techniques, which may include generating a depth map of the known workout space, tracking movement through a sequence of frames, deep learning algorithms, trained neural networks, and other suitable approaches. The three-dimensional data 3356 is analyzed to determine user position information 3358 that may be used to determine an exercise stage, whether the user is properly performing the exercise, and/or other user information.

In some embodiments, two-dimensional video is captured using a single camera (e.g., camera 206) and converted into a three-dimensional space to create a three-dimensional representation including depth. In some embodiments, any suitable machine vision algorithm may be used for identifying anatomical points in a two-dimensional image of the user and converting the identified anatomical points to a three-dimensional space, including depth. In some embodiments, the machine vision algorithm uses a single camera and multiple frames of captured images to generate the three-dimensional representation. In some embodiments, the system uses known dimensions for the exercise space and mat within the field of view of the camera and can estimate a depth of a point, for example, by identifying a location on the mat where each foot is positioned, analyzing an estimated size difference between left and right anatomical segments, tracking a change in size of an anatomical segment based on a motion, a change in angle of the anatomical segment from the point of view of the camera, and through other suitable methods.

On the user side, computer vision tracks the user while performing the exercises. In some embodiments, a rules-based system is defined that tracks a user's key points and angles for an exercise (e.g., left elbow position, right knee position, knee/ankle angle, hip/knee angle, etc.). In some embodiments, the rules are determined by the instructors, based on feedback. For example, instructors may watch video of users exercising and provide verbal instruction. Those instructions are turned into rules based on key point angles and positions of the users. In some embodiments, the rules are determined automatically based on instructor feedback, the system learning proper technique and identifying deviations from the proper technique. For example, average measurements can be taken of numerous squat repetitions to generate ground truth key points for the exercise. Corrections may be triggered when a key point measurement fails to satisfy a threshold of acceptable performance of the exercise.

In some embodiments, the system tracks anatomical key points, such as where hands are located in an image, where shoulders are located in an image, etc. In some embodiments, 17 key points are used to track user movements. The system tracks key points, distances between key points and angles between key points to drive feedback.

In some embodiments, after an instructor uploads exercise content, the exercise content is manually reviewed to identify exercises (e.g., squats) and the timing in the uploaded video where the exercise is to be performed. With the identified exercise, the system can identify a reference ground truth for performing the exercise during that time. The user performs the exercises, the performance is captured on video, and the system tracks the users 17 key points and angles and distances between key points. All of the key point information is fed into a state machine, which manages transitions from stage to stage in an exercise. The correction feedback could be triggered, for example, when the user doesn't complete a stage.

Accordingly, exercise systems, methods, and media for presenting exercise videos with feedback are provided. In one example embodiment, a method (which may be performed by a system comprising a display device and processing system) comprises receiving a sequence of camera images of a user performing an exercise, identifying a plurality of anatomical points of the user from the sequence of camera images, and determining user performance information for the exercise based on an analysis of the plurality of anatomical points. The method may further comprise applying exercise performance rules to the user performance information, the exercise performance rules including criteria for satisfactory performance of the exercise and providing corrective feedback to the user for exercise performance rules that are not met by the user performance information.

In some embodiments, the exercise comprises a plurality of movement stages to be performed in sequence and the step of determining the user performance information for the exercise further comprises analyzing the plurality of anatomical points to determine whether the user has performed the movement associated with each of the movement stages of the exercise. The method may further comprise displaying exercise content associated with the exercise, wherein the sequence of camera images is captured while the exercise content is being displayed.

In some embodiments, the method further comprises displaying content for the exercise on a display device comprising a housing having a recessed portion and a camera arm configured to be inserted in the recessed portion of the housing in a first position and partially inserted in the recessed portion of the housing in a second position, and extending the camera arm to expose a camera on one end thereof to position the camera to capture the sequence of camera images while the exercise content is being displayed.

In some embodiments, the sequence of camera images comprises a sequence of two-dimensional images and identifying a plurality of anatomical points of the user further comprises identifying the plurality of anatomical points on the two-dimensional images, and the method further comprises converting the two-dimensional anatomical points to three-dimensional anatomical points. The method may further comprise identifying performance criteria for the exercise, including an angle and/or position of at least one anatomic point during a movement stage of the exercise, and tracking the plurality of anatomical points in three-dimensional space to identify whether the performance criteria for the exercise is met. The method further comprises providing corrective feedback to the user if the performance criteria for the exercise is not met.

In some embodiments, the exercise has associated ground truth values represented as a sequence of anatomical angles and/or positions, and wherein the user performance information comprises comparison results between the ground truth values for the exercise and the identified plurality of anatomical points of the user. The method may further comprise calculating a score for the user's performance of the exercise based on comparison results between the ground truth values for the exercise and the identified plurality of anatomical points of the user. A user interface may be presented that includes a summary of the exercise including feedback associated with the user's performance of the exercise.

In some embodiments, exercise content is generated through a process comprising receiving exercise content from an exercise content provider, identifying a sequence of exercises within the exercise content and associated timing information for each exercise, identifying, for each exercise, at least one correction, and presenting a user interface to the exercise content provider for verification of the identified exercises, timing information, and at least one correction. The exercise content may be further generated through a process comprising receiving, from the exercise content provider, verification of identified exercises, timing information and at least one correction, receiving a user feedback message from the content provider for the at one correction, and storing the exercise content including identified exercises, associated timing information, identified corrections, and the user feedback message.

In some embodiments, the exercise is a mat-based exercise, a weightlifting exercise, a treadmill exercise, a cycling exercise, and/or a rowing exercise.

In another example embodiment, a system comprises a display device configured to displaying exercise content, the display device comprising a housing, a display screen on a front portion of the housing and a recessed portion on a back portion of the housing, and a camera arm configured to be inserted in the recessed portion of the housing in a first position and partially inserted in the recessed portion of the housing in a second position. The camera is further configured to extend to expose the camera on one end thereof to capture the sequence of camera images of the user performing the exercise while the exercise content is being displayed.

In some embodiments, the display device is further configured to receive a sequence of images from the camera of a user performing an exercise, wherein the sequence of images comprises two-dimensional images, wherein the display device is further configured to identify a plurality of anatomical points of the user on the two-dimensional images, and wherein the method further comprises converting the two-dimensional anatomical points to three-dimensional anatomical points.

In some embodiments, the system further includes a mat, free weights, a weight machine, a treadmill, a stationary bike, and/or a rowing machine. The camera arm may comprise magnetic components and an electronic communications port, and wherein the camera arm is detachable from the housing and adapted to attach to the housing via a magnetic connection with a corresponding magnetic element of the housing; and wherein the electronic communications port is configured to align with a corresponding port of the housing for electronic communications with the display device. The display device may be configured to display a dashboard screen to the user after an exercise session, including exercise corrections and highlights of the user's performances during the exercise session.

Although the various embodiments have been described and illustrated in the foregoing disclosure, it is understood that the present disclosure has been made only by way of example, and that numerous changes in the details of implementation can be made without departing from the spirit and scope of the present disclosure. Features of the disclosed embodiments can be combined and rearranged in various ways.

1. A method performed by an exercise system configured to display exercise content, the method comprising:
displaying exercise content associated with an exercise to be performed by a user; receiving a sequence of camera images of a user performing the exercise;
identifying a plurality of anatomical points of the user from the sequence of camera images;
determining user performance information for the exercise based on an analysis of the plurality of anatomical points;
applying exercise performance rules to the user performance information, the exercise performance rules including criteria for satisfactory performance of the exercise; and
providing feedback to the user based on the application of exercise performance rules to the user performance information.

2. The method of claim 1, wherein the exercise comprises a plurality of movement stages to be performed in sequence; and
wherein determining the user performance information for the exercise further comprises analyzing the plurality of anatomical points to determine whether the user has performed the movement associated with each of the movement stages of the exercise.

3. The method of claim 1, wherein the sequence of camera images is captured while the exercise content is being displayed.

4. The method of claim 1, further comprising:
displaying content for the exercise on a display device comprising a housing having a recessed portion and a camera arm configured to be inserted in the recessed portion of the housing in a first position and partially inserted in the recessed portion of the housing in a second position; and
extending the camera arm to expose a camera on one end thereof to position the camera to capture the sequence of camera images while the exercise content is being displayed.

5. The method of claim 1, wherein the sequence of camera images comprises a sequence of two-dimensional images;
wherein identifying a plurality of anatomical points of the user further comprises identifying the plurality of anatomical points on the two-dimensional images; and
wherein the method further comprises converting the two-dimensional anatomical points to three-dimensional anatomical points.

6. The method of claim 5, further comprising identifying performance criteria for the exercise, including an angle and/or position of at least one anatomic point during a movement stage of the exercise; and
tracking the plurality of anatomical points in three-dimensional space to identify whether the performance criteria for the exercise is met.

7. The method of claim 6, further comprising providing corrective feedback to the user if the performance criteria for the exercise is not met.

8. The method of claim 1, wherein the exercise has associated ground truth values represented as a sequence of anatomical angles and/or positions, and wherein the user performance information comprises comparison results between the ground truth values for the exercise and the identified plurality of anatomical points of the user.

9. The method of claim 8, further comprising calculating a score for the user's performance of the exercise based on comparison results between the ground truth values for the exercise and the identified plurality of anatomical points of the user; and
displaying a feedback screen associated with the user's performance of the exercise, including the score as a measure the user's performance.

10. The method of claim 1, further comprising displaying exercise content for the exercise on a display device; and
wherein the exercise content is generated through a process comprising:
receiving exercise content from an exercise content provider;
identifying a sequence of exercises within the exercise content and associated timing information for each exercise;
identifying, for each exercise, at least one correction; and
presenting a user interface to the exercise content provider for verification of the identified exercises, timing information, and at least one correction.

11. The method of claim 10, wherein the exercise content is further generated through a process comprising:
receiving, from the exercise content provider, verification of identified exercises, timing information and at least one correction;
receiving a user feedback message from the content provider for the at one correction; and
storing the exercise content including identified exercises, associated timing information, identified corrections, and the user feedback message.

12. The method of claim 1, wherein the exercise is a mat-based exercise, a weightlifting exercise, a treadmill exercise, a cycling exercise, and/or a rowing exercise.

13. An exercise system comprising:
a display device configured to display exercise content associated with an exercise to be performed by a user;
a processing system configured to:
receive a sequence of image frames of a user performing the exercise;
identify a plurality of anatomical points of the user from the sequence of image frames;
determine user performance information for the exercise based on an analysis of the plurality of anatomical points.
apply exercise performance rules to the user performance information, the exercise performance rules including criteria for satisfactory performance of the exercise; and
provide feedback to the user based on the application of exercise performance rules to the user performance information.

14. The exercise system of claim 13, wherein the exercise comprises a plurality of movement stages to be performed in sequence; and
wherein the processing system configured to determine user performance information for the exercise further comprises analyzing the plurality of anatomical points to determine whether the user has performed the movement associated with each of the movement stages of the exercise.

15. The exercise system of claim 13, wherein the display device further comprises a housing having a recessed portion and a camera arm configured to be inserted in the recessed portion of the housing in a first position and partially inserted in the recessed portion of the housing in a second position; and
a camera disposed on one end of the camera arm such that when the camera arm is in the second position the camera is in position to capture the sequence of image frames while the exercise content is being displayed.

16. The exercise system of claim 13, wherein the sequence of image frames comprises a sequence of two-dimensional images;
wherein identify a plurality of anatomical points of the user further comprises identifying the plurality of anatomical points on the two-dimensional images; and
wherein the processing system is further configured to convert the two-dimensional anatomical points to three-dimensional anatomical points.

17. The exercise system of claim 16, wherein the processing system is further configured to:
identify performance criteria for the exercise, including an angle and/or position of at least one anatomic point during a movement stage of the exercise; and
track the plurality of anatomical points in three-dimensional space to identify whether the performance criteria for the exercise is met.

18. The exercise system of claim 17, wherein the processing system is further configured to provide corrective feedback to the user if the performance criteria for the exercise is not met.

19. An exercise system comprising:
a display device configured to displaying exercise content, the display device comprising:
a housing;
a display screen on a front portion of the housing and a recessed portion on a back portion of the housing; and
a camera arm configured to be inserted in the recessed portion of the housing in a first position and partially inserted in the recessed portion of the housing in a second position;
wherein the camera arm is further configured to extend to expose a camera on one end thereof to capture a sequence of camera images of a user performing the exercise while the exercise content is being displayed.

20. The exercise system of claim 19, wherein the sequence of images comprises two-dimensional images; and
wherein the display device is further configured to identify a plurality of anatomical points of the user on the two-dimensional images, convert the two-dimensional anatomical points to three-dimensional anatomical points.

21.-23. (canceled)
